(19) United States
(12) Patent Application Publication
(10) Pub. No.: US 2014/0249612 A1
(43) Pub. Date: Sep. 4, 2014

(54) MRI COMPATIBLE LEADS FOR A DEEP BRAIN STIMULATION SYSTEM

(71) Applicants: Giorgio Bonmassar, Lexington, MA (US); Emad Eskandar, Nahant, MA (US)

(72) Inventors: Giorgio Bonmassar, Lexington, MA (US); Emad Eskandar, Nahant, MA (US)

(21) Appl. No.: 14/279,540

(22) Filed: May 16, 2014

Related U.S. Application Data
(63) Continuation-in-part of application No. PCT/US2013/063223, filed on Oct. 3, 2013.
(60) Provisional application No. 61/744,847, filed on Oct. 4, 2012.
(60) Provisional application No. 61/784,474, filed on Mar. 14, 2013.

(51) Int. Cl. A61N 1/08; A61N 1/05

(52) U.S. Cl. CPC A61N 1/08; A61N 1/0534; A61N 2001/086
USPC 607/116; 29/874

(57) ABSTRACT

A lead including a liquid crystal polymer including conductive particles dispersed therein. The lead may be adapted to conduct direct current for deep brain stimulation treatment or for use in other in vivo medical devices, while limiting the heat in implants in implants when exposed to MRI environments. Related methods of making the lead are also provided.

### CROSS-REFERENCE TO RELATED APPLICATIONS

This application is a continuation-in-part patent application of PCT application no. PCT/US2013/063223 entitled “MRI Compatible Leads for a Deep Brain Stimulation System” filed on Oct. 3, 2013 which claims the benefit of both U.S. Provisional Patent Application No. 61/744,847 filed on Oct. 4, 2012 and U.S. Provisional Patent Application No. 61/784,474 filed on Mar. 14, 2013, the contents of all of these applications are incorporated by reference for all purposes as if set forth in their entirety herein.

### STATEMENT OF FEDERALLY SPONSORED RESEARCH OR DEVELOPMENT

Not applicable.

### BACKGROUND

The present invention relates to brain stimulation systems. In particular, this invention relates to leads for brain stimulation systems that are compatible with magnetic resonance imaging.

Deep brain stimulation (DBS) is a surgical treatment in which an implanted medical device is used to regulate abnormal impulses or affect certain chemicals or cells in the brain. Typically this implanted medical device includes a brain pacemaker, often implanted under the skin in the upper chest, that sends electrical impulses to specific parts of the brain. DBS in select brain regions has provided therapeutic benefits for otherwise treatment-resistant movement and affective neurological disorders such as chronic pain, Parkinson's disease, tremors and dystonia. Deep brain stimulation is also being studied as an experimental treatment for epilepsy, cluster headaches, Tourette syndrome, chronic pain, stroke recovery, hypertension, Alzheimer's disease, addiction disorders, obesity, brain injury, minimally conscious states, anorexia, tinnitus and major depression.

Many patients with DBS would benefit from regular magnetic resonance imaging (MRI) examinations, as MRI is the preferred diagnostic tool for monitoring structural changes in the brain and for diagnosing injury due to trauma or evaluate comorbidities (for example, stroke and cancer, among others). Whole-body MRI examination is used in many common disorders including cancer, cardiovascular disease and trauma. Moreover, functional MRI could be potentially useful to study the effects of electrical stimulation of the basal ganglia.

Unfortunately, it has been found that magnetic MRI may cause deep brain stimulation (DBS) electrodes to become excessively hot and seriously damage adjacent brain tissue. Commercially-available DBS leads utilize crude metal wire, which act as antenna for the MRI radio frequency (RF) waves that are used during imaging to elicit signals from the brain tissue being imaged. The applied RF field induces currents along the conductive leads that can increase the RF power deposition at the distal tip of the leads and potentially result in excessive local heating at the electrode. To provide an appreciation of the potential heating effect, temperature increases up to 30° C. were recently recorded on the electrodes of a DBS implant in an ultra-field MRI (at 9.4 T). Furthermore, FIG. 1B illustrates a map of specific absorption rate deposition from an electromagnetic simulation in which a sharp increase is predicted at a tip of an electrode in a brain.

In view of this risk, the Food and Drug Administration (FDA) has only approved the use of metal wire in DBS leads by restricting their use to transmit head-only coils and fields up to 1.5 T. However, by excluding the use of transmit body coils (by far the most common type of transmit coils used) and 3 T or greater systems, MRI is severely limited as a diagnostic tool in patients with DBS implants. For example, the conditions under which the Medtronic MR-conditional DBS system can be used are extremely restrictive: it is possible to scan patients with DBS implants only with a transmit head coil and with a whole-head averaged Specific Absorption Rate (SAR) of only 0.1 W/kg. Notably, the normal operating mode allows whole-head SAR of 3.2 W/kg. Transmit body coil, 3 T systems, and the state-of-the-art MRI multichannel transmit coils are all contraindicated.

Thus, while there are over 75,000 patients with DBS implants worldwide, only approximately one patient in twenty is assessed with MRI because of the restrictions on its use. More generally, because of safety concerns with the heating of leads, each year approximately 300,000 patients with implants such as implantable cardioverter defibrillator (ICD), pacemaker, and DBS as well as guidewires, such as ablation catheters, are denied MRI.

Such restrictions are not misplaced, as two cases of serious, permanent neurological injury related to the antenna effect and excessive heating of DBS leads during MRI have been reported including one patient that experienced a temporary dystonia and another patient that developed a permanent hemiparalysis. In one case, a patient with two bilateral implants underwent a routine MRI of the lumbar spine and the high RF-induced currents generated by the body coil on the DBS implants (notably of different length) produced edema near one of the implants, illustrated in FIG. 1A, resulting in consequent paralysis.

MRI adverse events in DBS patients are anticipated to continue to be problematic because of an increasing number of scans. Further, as MRI is becoming available even in the smallest rural or private medical centers where MRI Safety educational training to clinical and technical personnel in MRI sites may not be readily available.

Hence, a need exists for an improved DBS system that is fully MRI-compatible.

### SUMMARY OF THE INVENTION

An improved lead is disclosed. According to one aspect, the lead is a lead for a deep brain stimulation system in which the lead is adapted for electrical communication with a neurostimulator and extends to a distal tip for attachment to at least one electrode. The lead comprises a lead wire comprising a liquid crystal polymer including conductive particles dispersed therein.

The liquid crystal polymer may include polyesterpolyarylate fibers. The conductive particles may nanoparticles such as, for example, gold nanoparticles having an average diameter of 4 to 5 μm or carbon nanoparticles having an average diameter of less than 1 μm. However, other polymers, conductive particles, and sizes of particles might be used in order to provide the DBS and MR compatible leads. The conductive particles may be melt polymerized with the liquid crystal polymer to disperse the conductive particles throughout the liquid crystal polymer. Other materials may also be used as fillers (apart from conductive particles), such as fiberglass, to improve physical characteristics.

The conductive nanoparticle or micro particles are also non-ferromagnetic and biocompatible, and may include carbon, gold, platinum, titanium, niobium, tantalum, cobalt-chromium, cobalt, stainless steel, chromium and zirconium. Alloys of non-ferromagnetic biocompatible nanoparticles and microparticles may also be used.

It is also contemplated that coated nano- or micro-particles that are non-ferromagnetic, biocompatible and conductive may be also used as conductive filler.

A coating can be introduced to improve other physical characteristics, such as: thermal conductivity, thermal expansion, corrosion resistance, density, elongation, fatigue endurance limit, melting and boiling points, hardness, impact energy, modulus of elasticity, corrosion behavior, Poisson's ratio, reflectance, shear strength, specific gravity, electrical conductivity, electrical permittivity, tensile strength, yield strength, Young's modulus, and/or fracture toughness. Chemical compounds can be formed also to change the sintering temperature, surface porosity, and color, and, of particular importance in implantable lead to improve biocompatibility.

It is contemplated that in some forms of the invention, the liquid crystal polymer fibers or the lead implant itself may be coated with gold or other biocompatible metal (such as, for example, platinum) using either vacuum coating, sputtering, ion beam assisted/induced deposition, cathodic arc deposition, electrospray and matrix-assisted laser desorption/ionization. This can help to disperse the conductive phase in the polymer or liquid crystal polymer.

The lead wire has abrupt variations in resistance over its length which can prevent undesirable RF heating from occurring at the ends of the leads. This means the improved leads may be implanted, used for treatment in DBS systems or the like, and moreover imaged at the currently contraindicated MR operating conditions for existing leads. As one example, when the lead is implanted in a patient and subjected to radio frequency waves in an MRI device, the lead may not heat more than 2 degrees Centigrade in an applied field of 3 Telsa.

The lead may also have a packaging similar to existing leads (for example, may be approximately 1.3 mm in diameter). An insulating outer coating, such as a polyurethane coating, may be received on the lead wire. The lead may include multiple bundles (for example, four bundles), in which each bundle includes a lead wire that with an insulating outer coating, and these multiple bundles are packaged together in a single lead. In this instance, each of the multiple bundles may be received in additional liquid crystal polymer (to strengthen the lead) and may have an insulating sheathing for insulation as well as biocompatibility.

Based on the disclosed design, the lead wire may be adapted to conduct direct current for deep brain stimulation treatment, while remaining substantially transparent in clinically-applicable MR environments, including those which are currently contraindicated but highly valuable for treatment of patients.

According to one aspect, a deep brain stimulation device is provided including a neurostimulator, an electrode, and a lead (as described above or elsewhere in this disclosure) in which the lead places the neurostimulator and the electrode in electrical communication with one another.

However, it will be appreciated that the lead might be useable in other non-DBS systems such as, for example, pacemakers. Other applications may include, but are not limited to: functional electrical stimulation used in spinal cord injury, back pain and stroke; median nerve stimulation used in epilepsy; cortical stimulation used in epilepsy, brain computer interface and to promote awakening from vegetative state or coma; sub-epidermal Electric Stimulator Implant for migraine; occipital nerve stimulation for occipital neuralgia and chronic migraines; neuromodulation for managing urinary function or to control chronic pain and Sacral nerve stimulation to control the bladder; cochlear implants, gastric neurostimulator implant; and implantable bone growth stimulation.

According to yet another aspect, an MR-compatible lead is disclosed generally in which a lead wire includes a polymer (which may be a LCP or another biocompatible polymer such as for example, but not limited to, PEEK, PAI, PEI, PPSU, POM and implantable grade ultra high molecular weight polyethylene which all are currently approved for long-term implantation) with a conductive phase (either particles dispersed therein or a conductive coating on a sheet that is rolled as described below). Again, the lead may be able to transmit a direct current signal while being substantially MR transparent in clinically applicable MR environments.

According to still another aspect, a method of making a lead is disclosed. A liquid crystal polymer and conductive particles are mixed to form a mixture in which the conductive particles are dispersed in the liquid crystal polymer and, from the mixture, a lead wire is formed. This forming may involve, in some embodiments, extruding the mixture to similarly orient fibers of the liquid crystal polymer in a direction of extrusion.

It is contemplated that the improved lead may not be limited to conductive particles dispersed in a liquid crystal polymer, but may include other polymers. These polymers may include, but are not limited to, PEEK, PAI, PEI, PPSU, POM and implantable grade ultra high molecular weight polyethylene. Likewise, the conductive phase may not be limited to conductive nanoparticles of gold and carbon, but may include (but are not limited to) gold, platinum, titanium, niobium, tantalum, cobalt-chromium, cobalt, stainless steel, chromium and zirconium.

According to one embodiment, a wire may be built from a wrapped up, rolled up, or coiled thin sheet of polymeric material. In one form, the sheet may be approximately 25 microns thick. This rolled design is also workable because the DBS electrode needs to be stiff and it is a straight lead. The polymer sheet is transformed into a conductor before it is coiled by either dispersing fine non-ferromagnetic, biocompatible and conductive particles into a molten form of the sheet or by coating the sheet. Coating may be performed either by vacuum coating or by applying a fine non-ferromagnetic, biocompatible and conductive medium that is then cured leaving a conductive surface. This deposited coating creates a “primer” layer and conductivity can then be controlled by building up thickness by resistance or timing controlled electrical metal fine non-ferromagnetic, biocompatible and conductive electro plating. Different metals or carbon can be used in electroplating with different conductivity to match the different RTS layers. The metals may include: gold, platinum, titanium, niobium, tantalum, cobalt-chromium, cobalt, stainless steel, chromium and zirconium.

These and other advantages of the present invention will be apparent from the description below and the accompanying drawings. While a preferred embodiment is described and depicted, it should be understood that this disclosure is not made by way of limitation.

### BRIEF DESCRIPTION OF THE DRAWINGS

It should be understood that the drawings are provided for the purpose of illustration only and are not intended to define the limits of the invention. The present invention is not limited to the precise arrangements and instrumentalities shown in the drawings, and the drawings are not necessarily to scale, emphasis instead being placed upon illustrating the principles disclosed herein, wherein:

FIG. 1A illustrates lesions that have occurred after routine MRI recording on patient with a DBS implant and a hemorrhagic lesion that has formed near the electrode at the tip of the implant where current is delivered for stimulation. This hemorrhage was attributed to RF-heating of the electrode. In FIG. 1B, results of electromagnetic simulations are illustrated predicting an increase of power deposition near the tip of the electrode.

FIG. 2 illustrates a schematic of a DBS system implanted in a patient.

FIG. 3 illustrates liquid crystal polymers (LCP) fibers and compares them to polyester polymer molecules. In FIG. 3A, LCP polymer molecules are illustrated as stiff structures organized in ordered rod-like domains, whereas in FIG. 3B the polyester polymer molecules are distributed randomly and are flexible molecular chains. In FIG. 3C, the chemical formula of an exemplary LCP fiber is illustrated.

FIG. 4A illustrates an InkCap which is a high-resistive 32-electrode EEG cap based on polymer thick film (PTF) tested for safety with magnetic fields up to 7 T. In the top image of FIG. 4B, induced currents are simulated for standard copper and, in the bottom image of FIG. 4B, resistive leads are simulated using FDTD algorithm with a multi-structure 1 mm³ resolution. The red in the color bar code corresponds to 0 dB=1000 A/m².

FIG. 5A illustrates an RF impedance measurements setup with an Agilent 16093/A Binding Post fixture. FIG. 5B illustrates an equivalent electrical circuit is illustrated with an electrical length of 0.34 cm, L=1.8 nH, and C=1.8 pF. The fixture required double calibration (standard and as an external port) for the electrical length and residual compensation. FIG. 5C illustrates resistivity measurements taken with a commercial LCR meter at frequencies between 100 Hz and 200 kHz on traces of 5 different lengths are illustrated.

FIGS. 6A and 6B illustrate resistivities of the traces over the frequency range of 100-300 MHz. FIGS. 6D and 6E illustrate reactances over the frequency range of 100-300 MHz. FIG. 6C illustrates the return loss of the stripline average plus or minus standard error. FIG. 6F Smith Charts of the traces (as the Smith Charts were similar we only show one of the sets).

FIG. 7A provides a plot of resistances, currents and cost function per iteration. FIG. 7B illustrates an HFSS model with the ASTM phantom, DBS leads and the body coil.

FIG. 8 illustrates, on the top left, the ASTM phantom (see ASTM 2182-11); on the top right, the five channel phase array receive-only coil inside a body coil, a phantom, and DBS leads; and, on the bottom, the circuit of lead current detection based on a design for RF current detection in pace maker leads during MRI. The circuit consist of a light-emitting diode (LED) supplied by a rectifier bridge, which transforms the RF pulse into a light signal and then measured optically using the plastic optic fiber. The temperature of the tip of the DBS electrode set is also be measured optically with a Fluoroptic thermometer.

FIG. 9 illustrates, in the left two images, a high-resolution (350 μm isotropic) model of rhesus-monkey with 7 anatomical structures labeled. In the right two images, SAR and temperature maps are provided. Temperature results are normalized to 1.15 W/kg peak SAR.

FIG. 10 shows the GS100 Imtram PTF Ink Transfer Unit with a PTF 64-electrodes array.

FIG. 11 illustrates a 3D reconstruction of DBS electrode traversing caudate and nucleus accumbens.

FIG. 12 shows the RTS design and simulation setup. In FIG. 12A, a schematic of the metallic wire (diameter d, electrical conductivity σ) and the two-layer RTS design (electrical conductivity σ₁ and σ₂, permittivity ∈₁ and ∈₂, length L₁ and L₂) used for the study are shown. In FIG. 12B, the equivalent circuit used to model the RTS implant with four sections is illustrated including the stimulator, two layer transmission line, and electrode/tissue interface. The incident RF field induces currents along the implants, which are reflected depending on neighboring sections mismatched impedance (Z₀, Z₁, Z₂, and Z_{L}). The resulting voltage amplitude at each interface (V₀, V₁, and V₂) was generated by the induced current. FIG. 12C compares RF-induced currents along the two types of leads (that is, wire lead verses RTS lead). The current in the metallic conductor forms a standing wave with high peaks in amplitude (I_{W}); conversely, the effect of RTS design is two-fold as it reduces the average induced currents (I_{RTS}) along the implant by worsening the antenna performance, and reduces the induced current at the electrode (AI) by introducing scattering within the implant. IN FIG. 12D, a CAD Model used in the FEM simulations is shown, including a 16-leg high-pass birdcage body coil with RF shield, coil former or container and ASTM phantom. In FIG. 12E, lead placement inside the model of the phantom is shown. The lead model was placed in a volume with high electric field amplitude.

FIG. 13 illustrates the electromagnetic energy and thermal characterization of RTS lead in phantom. Numerical simulation results at 128 MHz were calculated with finite element method using the geometry shown in FIG. 12D and with either a single-electrode Pt/Ir wire or a RTS lead. In the top row, 10g-averaged SAR in the ASTM phantom without lead (left), with RTS profile that was selected for prototype manufacturing (middle), and with the Pt/Ir wire (right). Values normalized to whole-body SAR of 2 W/kg. In the bottom row, corresponding temperature maps in the three cases for 15 minutes of continuous SAR exposure. Simulations showed that the RTS design was transparent to the incident RF and generated similar temperature increase (up to 1.3° C.) compared to the ASTM phantom without lead. By contrast, the Pt/Ir wire generated a temperature increase up to 64° C. near the electrode.

FIG. 14 illustrates how RTS design can be optimized be calculation. FIG. 14A is a schematic of the two-layer RTS design (electrical conductivity σ₁ and σ₂, permittivity ∈₁ and ∈₂, length L₁ and L₂ used for the study. FIG. 14B shows the calculated 10 g-averaged SAR inside the phantom at a distance of 0.1 mm from the electrode obtained varying the length (L₂) of the second section. Plots include different conductivity ratios for the two layers. In all cases, the total resistance of the lead was R=400Ω. FIG. 14C shows 10 g-averaged SAR in the same point obtained varying the total resistance of the lead. Plots include four combinations of conductivity ratios of the two layers and length L₂ of the second section. FIG. 14D shows maximum inductance of the RTS varying the total resistance of the lead. Plots include five combinations of conductivity ratios of the two layers and length L₂ of the second section. FIG. 14E shows amplitude of induced current inside the lead with the Pt/Ir wire, with the RTS lead selected for prototype manufacturing (right) and in the corresponding volume of the ASTM phantom without lead. The RTS lead allowed for a 37-fold decrease in induced current at the electrode (x=0). In all cases, the total length of the leads was 40 cm.

FIG. 15 shows the experimental temperature measurements. In FIG. 15A, the RTS leads manufactured with a complete PtIr electrode are depicted. In FIG. 15B, the temperature experiments are illustrated showing the ASTM phantom in a 3 T system on the left, lead placement inside the phantom in the middle, and detail showing the temperature sensors and the RTS and commercial lead electrodes on the right. In FIG. 15C, temperature measurements are illustrated at three different position within the phantom without lead, with 3389 lead, and with the RTS lead.

FIG. 16 illustrates the results of the battery consumption test. In the top row, FIG. 16A depicts the configuration with RTS lead and FIG. 16B depicts the commercial lead connected to commercial DBS IPG system. FIGS. 16C and 16D show the RTS leads and the commercial leads were immersed in physiologic solution, respectively. Below the depictions of the test setup are the results of the battery consumption and impedance profiles over the four weeks testing period in which the number on the X axis indicates the number of days.

FIG. 17 is a histogram showing worst case scenarios estimated by numeric simulation. The 10 g-averaged SAR at the Larmor frequency varies with: shape (i.e., cylindrical vs thin), and conductivity (homogeneous vs. RTS). These results illustrate that cylindrical geometries produce larger SAR than thin geometries. RTS showed lower SAR than thin-flat lead with homogeneous conductivity.

FIG. 18 illustrates the distribution of the magnitude of the electric field (left) at the Larmor frequency in an ASTM Phantom and in the same coordinate system is shown (right) the lead placement.

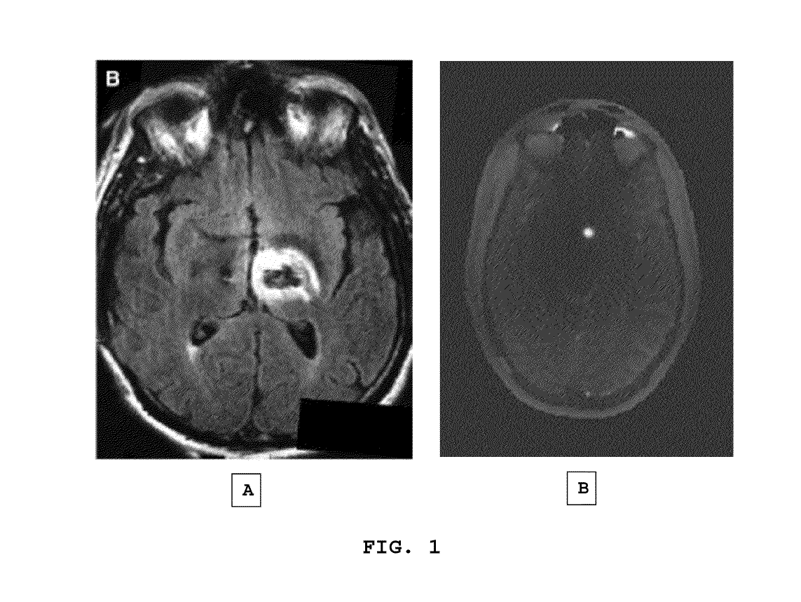

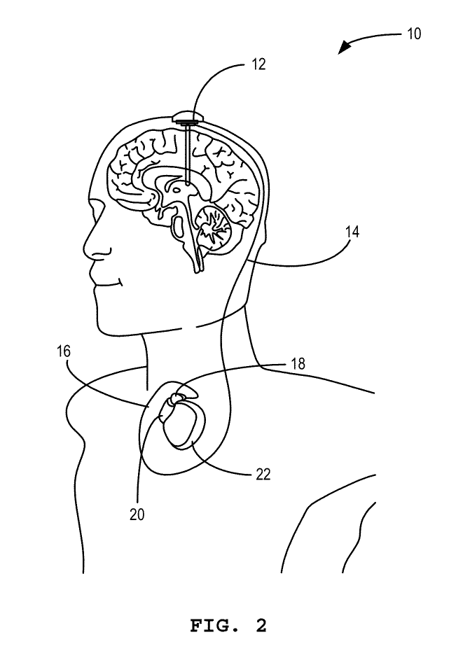

### DETAILED DESCRIPTION

As outlined above, both electromagnetic simulations in human head models with implants and case studies have shown that traditional leads implanted in the brain can produce local heating during magnetic resonance imaging.

To overcome the problems with existing leads, in this disclosure, we present new neural prosthetic leads that can be used as MR-conditional intracranial implants in human subjects. These leads, which may be used in DBS systems or the like, are based on resistive tapered striplines (RTS) technology such as is found in the literature. See, for example, Bonmassar G. “Resistive Tapered Stripline (RTS) in Electroencephalogram Recordings During MRI.” IEEE Trans on Microw Theory and Tech. 2004; 52(8):1992-8. The RTS-type lead design reduces the RF-induced currents along the DBS implant as well as the related increase of RF power deposition and potential tissue heating near the tip of the leads. As used herein, the improved leads may be referred to, for example, as “RTS leads,” “LCP leads,” “DBS leads”, or simply “leads.” In the instance in which the improved leads are being compared to conventional leads, or are being prototyped, it will be so indicated by the context of the detailed description.

The principle behind the RTS design can be best understood by recalling oceanic science, where standing waves (called clapotis) are sometimes formed. Special constructions called caisson-type breakwaters have been reinforced with wide rubble-mound beams to break up wave energy over some distance, preventing the formation of clapotis.

Similarly, the disclosed design incorporates a lead with abrupt variation of resistance along its length, which essentially breaks up the energy of the RF wave in the wire by scattering. Specifically, this disclosed design embeds RF transparency in a conductive liquid crystal polymer (LCP) and modifies it with a tapered dielectric structure in the form of nanoparticles. In some specific forms, the LCP material can have a high tensile strength and is up to five times stronger than the current leads, which occasionally fracture. Prototypes of these DBS leads can be built using current bench-level polymer thick film (PTF) technology.

The RTS leads are divided into segments with different unmatched impedances that allow reflecting back to the input parts of the incoming RF emitted from the MR transmit coil, thereby minimizing RF deposition into the patient. Conversely, the RTS-type structure reduces the low-frequency resistance (that is, the real part of the impedance) to preserve the battery life of the neurostimulator. The RTS-type structure of the leads allows for very low overall DC resistance of the leads using the novel materials and thus the novel DBS system will still have a standard battery life, which cannot be achieved using traditional purely resistive leads.

This improved lead design significantly impacts the neural prosthetics field by creating a new state-of-the-art lead for a medical implant that is compatible with a wider range of MRI use. As the disclosed leads achieve a high degree of RF-transparency, maintain the current DBS lead form factor, and containing only minimal amount of metal, this allows for the scanning of patients even under very broad conditions, presently absolutely contraindicated. These conditions include: the use MRI in normal operating mode (whole-body SAR of 2 W/kg, whole-head SAR of 3.2 W/kg), the use of 3 T or higher static fields, the use of RF transmit body coil, and the use of multichannel transmit coils.

This allows patients with DBS implants to benefit from the complete diagnostic benefits of MRI, including for example disease diagnosis in body soft tissues. This will have a high-impact on public health because, while MRI and non-soft tissue CT examinations are ranked by physicians as the most important technologies affecting their ability to treat patients, currently less than 5% of the patients with DBS benefit from MRI, and even then only a partial MRI given the recited restrictions on use.

It is contemplated that besides the FDA approved applications of Parkinson's disease, dystonia, and obsessive compulsive disorder, the proposed leads implementing RTS technology may be employed in future clinical applications of DBS including major depressive disorder, disorder and epilepsy and potentially, with further testing, in other active implants such as cardiac pacemakers which are implanted in hundreds of thousands of patients worldwide.

Moreover, the disclosed leads may offer other benefits unrelated to their improved MRI compatibility. For, example, these leads may be less susceptible to electromagnetic interference (EMI) from external RF sources such as for example, metal detectors, anti-theft systems and communication systems (for example, cell phones, RF towers).

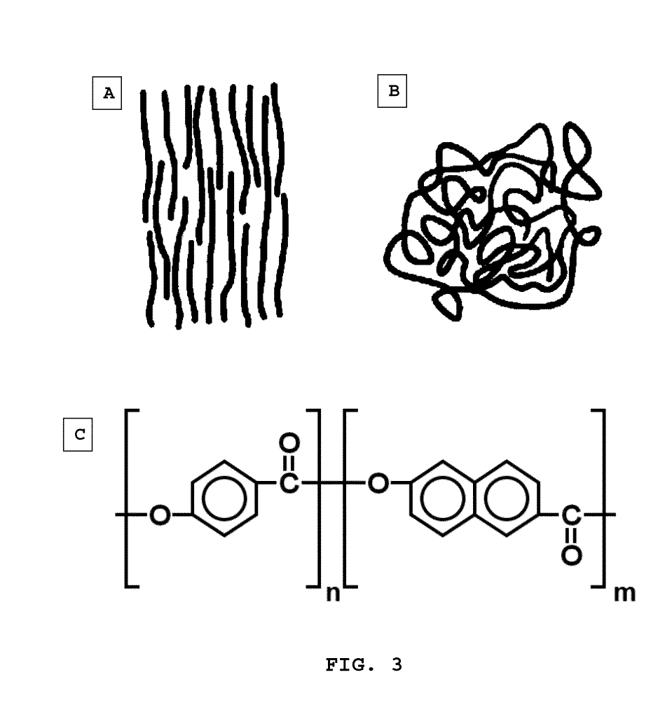

Referring particularly to FIG. 2, a DBS system 10 is illustrated including an electrode probe 12 that is capable of both stimulating populations of neurons and measuring single-unit neuronal activity. The probe 12 is typically implanted in a targeted area, for example, the subthalamic nucleus (STN), and connected to an insulated lead 14 that is passed under the skin of the head, neck, and shoulder and terminated at a neurostimulator 16. The neurostimulator 16 typically sits inferior to the clavicle and is programmed to operate the DBS system 10. A pulse generator 18, a controller 20, and battery pack 22 that powers the apparatus are all included in the neurostimulator 16.

Still referring to FIG. 2, in operation, the DBS system 10 acquires neuronal activity, or spike train, data with the electrode probe 12. This neuronal activity data is carried via the lead 14 to the neurostimulator 16 where it is processed by the controller 20. The controller 20 analyzes this data and predicts a responsive stimulation signal that will prevent future pathological neural events. The stimulation signal is generated by the pulse generator 18 and delivered via the lead 14 to the electrode probe 12, which administers the stimulation signal to the targeted area. It is contemplated that the response may inhibit the neuron, excite the neuron, or do nothing.

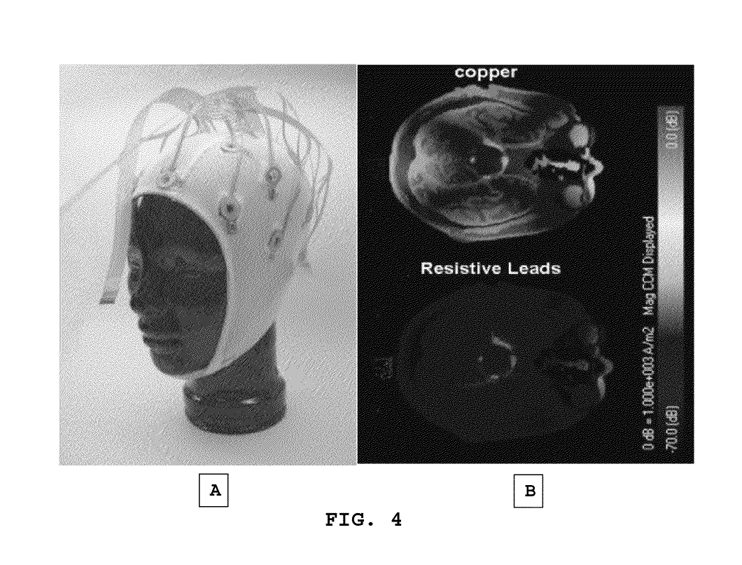

Turning now to FIG. 3, the LCP material is illustrated for the improved lead design. The LCP fiber in this instance is a polyesterpolyarylate fiber belonging to the class of aromatic polyesters and its chemical structure is illustrated in FIG. 3C. The fiber is based on HBA/HNA (that is, p-hydroxybenzoic/phydroxynapthodic acids) copolyesters, prepared by melt polymerization at 250°-280° for 4 hours. Comparing FIGS. 3A to 3B, the oriented LCP fibers are shown in comparison to randomly distributed polyester molecules. The LCP fibers possess unique properties, such as: high strength for mechanically biostable leads, excellent creep resistance to ensure long life of the chronic implants, high abrasion resistance to sustain the repetitive wire linear motion during subject's movement, excellent flex/fold characteristics optimal for bending reliability as will be outlined below, minimal moisture absorption for avoiding leaking/corrosion and improving biostability, excellent chemical resistance for biocompatibility, low coefficient of thermal expansion for lead fabrication, high dielectric strength for insulation (notable, since conductive particles will be mixed with the LCP fibers), outstanding cut resistance for avoiding electrical breaks in the implant lead, excellent retention properties for prolonged implant life, high impact resistance, and good shock absorbance for reducing potential neuroprosthetics leads damage during accidents. LCP fibers orientated as in FIG. 3A are five times stronger than steel and ten times stronger than aluminum. Outgassing tests show that LCP fibers perform well within parameters for medical applications including DBS. LCP may also offer decreased UV degradation for resilience to implant sterilization.

LCPs are extremely biocompatible given that they are exceptionally inert. LCPs are capable of withstanding most chemicals at elevated temperatures, including aromatic or halogenated hydrocarbons, strong acids, bases, ketones, and so forth. Chronically implanted electrodes often can provoke an immune reaction against them. Histopathology analysis often shows gliosis and spongiosis around the electrode track, which forms an encapsulation layer referred to as a “glial scar.”

Since LCPs are non-conductive, a doping step with gold nanoparticles (AuNPs) and/or carbon nanoparticles (CNPs) is added to the melt polymerization process of the LCP manufacturing in order to produce the material for the leads and to impart the conductive qualities used in DBS treatment. It is contemplated that other conductive materials may be used and, rather than being particles dispersed in a mixture with the polymer, they may be a conductive layer formed on a thin polymeric sheet that is subsequently rolled or coiled to form a wire lead. The target conductivities may be determined using electromagnetic simulations as outlined below. Depending on size, shape, and chemical surface of AuNPs, a layer of oxidation or a protein corona may occur. The potential problem is that the water-gold reduction (2 Au+3 H₂O→Au₂O₃+3 H₂) modifies the local pH and may generate inflammation in the surrounding tissue or by other mechanisms that ultimately may prompt an immune reaction.

Since any chemical element is toxic at high dose, it will be established that AuNPs and CNPs are not toxic at the low concentration at which they may leak in the tissue. A recent review suggests that AuNPs are biocompatible at low dose, as they are being evaluated as neurological drug delivery agents in clinical trials. Similarly, CNPs are considered safe in humans as they have been used in electrodes for decades (carbon black). To further ensure the biocompatibility of the proposed fibers, the AuNPs and CNPs are tested for biocompatibility in rats by the Charles River Laboratories International (CRL), Inc., Wilmington Mass. to test that the RTS leads do not have a worse glial scar/immune response when compared to the polyurethane used in the commercial DBS sets.

A numerical framework based on a combination of Finite Differences Time Domain (FDTD) and Finite Element Method (FEM) simulations are used to optimize the design of the RTS leads. Anatomically precise head models with implanted DBS leads with a multiscale resolution of 1-0.1 mm³ isotropic have been developed which allow for accurate geometrical modeling of the implanted leads as well as precise computation of 1 g- and 10 g-averaged SAR. The FDTD simulations have been validated with temperature measurements and have been shown to provide accuracy of 20% as predicted by the bioheat equation for whole-body SAR and phantom. However, the use of the whole-head SAR or even 10 g-averaged SAR as dosimetric parameter for safety profile with thin-wire (PEC) such as the RTS leads may be potentially inaccurate and local SAR should be considered instead. The bioheat equation predicts that minimizing the local SAR is equivalent to minimizing the tissue heating. The underlying assumption of lack of perfusion used for in vitro study represents a worst-case scenario for temperature changes at the distal tip and related tissue injury. Thus, the in-vitro temperature measurements are currently the only reliable safety validation tool with metallic implants as shown by several groups and are used to confirm the RTS simulations. FDTD simulations are performed using different number of RTS layers and the simulation is coupled with an optimization algorithm in FEM to estimate the ideal RTS parameters for each layer, such as length and conductivity.

The final optimization result of the combined FDTD/FEM simulations provides the specifications to build the leads using the PTF technology available at the Analog Brain Imaging Laboratory at Massachusetts General Hospital. The leads are tested at field strengths of 0.5 T, 1 T, 1.5 T and 3 T on the gold standard head and torso phantom specified by ASTM (ASTM 2182-11). Temperature measurements are performed with fluoroptic temperature probes, extensively used by the scientific community for use in MRI. Based on the simulations and prototype PTF leads, LCP fibers with RTS layers are then produced, by changing the concentration of AuNPs and CNPs as similarly done with the prototype PTF leads.

The overall resistance of each lead is preferably below 25% of the maximum electrodes/tissue contact impedance of the DBS system or more preferably below 10%. Additionally, the battery life of the simulator should preferably be able to last at least six months using the new lead design.

Specific examples of the processes used to develop, prototype, and test the improved leads for a DBS system are provided below. These examples are offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### Example I

In consideration of whether conductive polymer would produce image artifacts in the MRI, previous work and results using an InkCap were considered. See, Vasios et al. “EEG/(f)MRI measurements at 7 Tesla using a new EEG cap (“InkCap”).” Neuroimage. 2006; 33(4):1082-92.

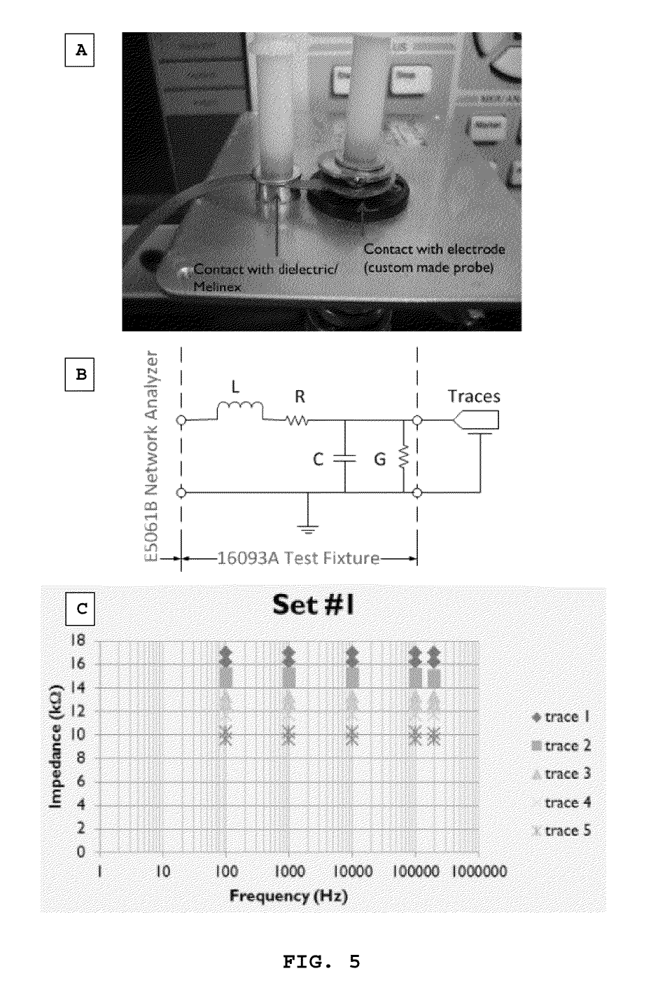

As illustrated in FIG. 4, Polymer Thick Film (PTF)-based leads have been successfully used in an InkCap for simultaneous EEG-fMRI recordings in human subjects at 7 T. The InkCap shown in FIG. 4A are made of conductive polymer microstrips measuring approximately 750 μm wide by 18 μm (±30%) thick. The resistance per unit length of the microstrips was 2 kΩ/m and their length varied between 35 and 56 cm. The resistivity of the microstrips was chosen in accordance with manufacturing constraints. The electrodes were Ag/AgCl-printed rings and two motion sensors were placed on the temporal regions of the cap.

This InkCap was tested in three different ways: FDTD simulations depicted in FIG. 4B, temperature measurements in an electrically conductive phantom head at 7 T, and EEG recordings during structural and functional MRI at 7 T in 12 healthy human volunteers. To compute electromagnetic fields and SAR, the XFDTD program (REMCOM Co., State College, Pa. —based on the FDTD algorithm) was used. All simulations were performed at the RF frequency of 300 MHz, corresponding to proton imaging with a static B₀ field of 7 T, and with a 16-rods birdcage coil.

Based the InkCap results, in which the substrate used was also a plastic-like LCP, it can be said with confidence that there will be no image artifacts from the use of LCP, and they will not be visible apart from the lack of MRI signal inside the RTS leads, given that plastics do not usually contain any water molecules (and hence have no MRI signal). However, MRI artifacts may arise if the gold nanoparticles are contaminated by ferromagnetic metals. To confirm the lack of ferromagnetic contaminants in the gold nanoparticles prior to lead fabrication, the nanoparticles may be tested first using MRI, for example.

### Example II

Polymer Thick Film (PTF)-based RTS prototypes are manufactured. The desired conductive ink with dielectric properties matching the values of the simulation are made at the AA Martinos Center at Harvard Medical School by mixing carbon, gold inks, and all traces are coated with dielectric coating. The printing is done on standard polyester film (Melinex®, DuPont Teijin Film, Chester Va.) substrate using a PTF deposition system (GS100 ITW Imtran, available from Haverhill, Mass.) equipped with micromanipulator as illustrated in FIG. 10 for precise RTS fabrication. Otherwise, screen printing technology can be used to create longer RTS traces.

Electrical impedance spectroscopy measurements were obtained to study two common PTF inks both at low frequency and in the frequency range of interest (32-128 MHz, the Larmor frequency range of 0.5 T-3 T). Traces were built by a PTF manufacturer (GM Nameplate of Seattle, Wash.) with a 32 mils width, resistivities as the ones of the Inkcap, and of five different lengths. All traces were silk screen printed on top of a Melinex® polyester film substrate (obtained from DuPont Teijin Films of Chester, Va.) using two different sets of conductive ink mixtures each having its own dielectric ink layer on top. The two sets were based on a different binder system, namely on a two parts epoxy for the first set (set #1) and acrylic for the second set (set #2). Set #1 was a mixture of Ag-based PTF ink, (CI-1001, from Engineered Conductive Materials LLC of Delaware, Ohio; hereafter “ECM”), and C-based PTF ink (CI-2001, ECM). The electrodes in set #1 were made out of Ag/AgCl based PTF (CI-4006, ECM), and the dielectric ink PTF (DI-7510, ECM) was UV-cured ink while all the others were temperature-cured. Set #2 was also mixture of Ag based-PTF ink [Electrodag 725 A (6S-61) from Acheson-Henkel Electronic Materials LLC, of Irvine, Calif.; hereafter “Acheson”] and C-based PTF ink (Electrodag 440B, Acheson). The electrodes in set #2 were made out of Ag/AgCl based PTF, (Electrodag 7019, Acheson), and the dielectric ink PTF (Electrodag PF-455B, Acheson), was UV cured ink while all the others were temperature cured.

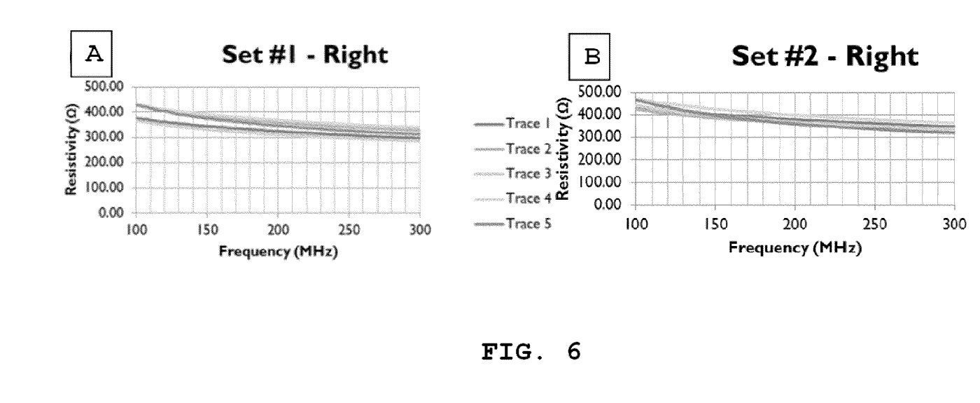

The low frequency measurements were performed using a BK Precision 889 Bench LCR/ESR Meter. The results, which are illustrated in FIG. 5C, suggest that there is very low impedance dispersion over the range of frequencies. That is, the response of the magnitude of the impedance is flat in frequency (100 Hz-200 kHz). All the RF measurements were performed using a network analyzer (E5061B obtained from Agilent Technologies, Inc., Santa Clara, Calif.) and the Agilent 16093/A binding post fixture, which required a double calibration and which is illustrated in the photo of FIG. 5A and the equivalent electrical circuit of FIG. 5B.

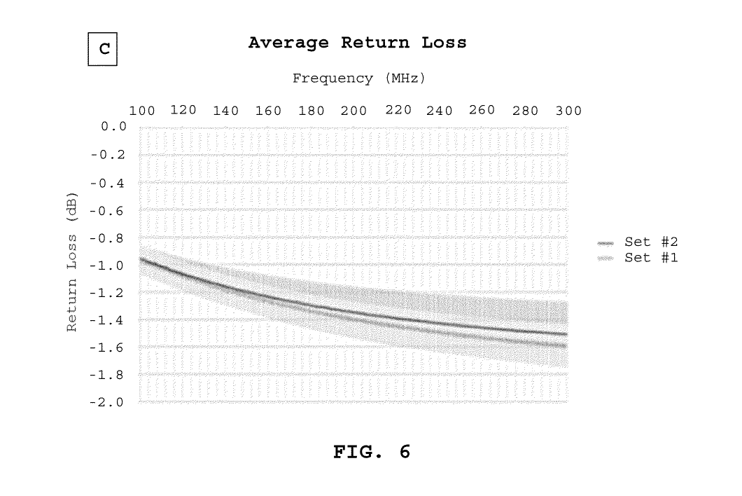

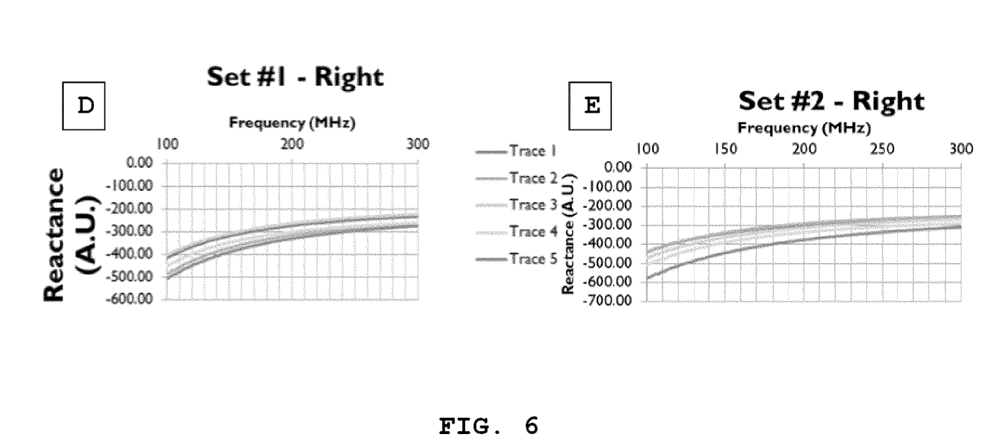

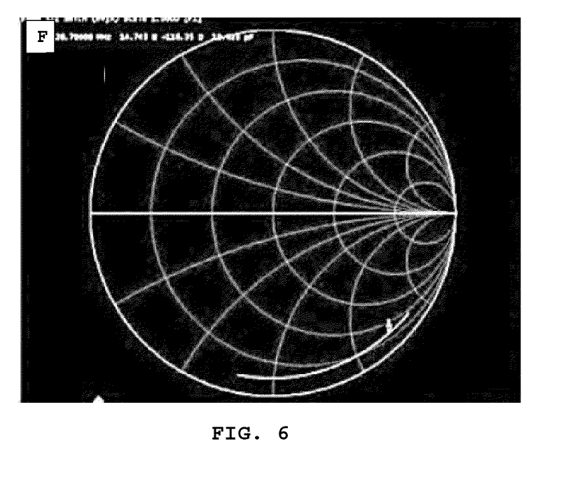

The stripline resistivity results are illustrated in FIGS. 6A and 6B for sets #1 and #2, respectively, and are very similar to the resistivity measurements of the traces done at low frequency. The stripline measurements were not direct since they also measure the effect of the Melinex® film substrate and the dielectric ink. At 128 MHz (that is, the frequency corresponding to 3 T) both sets show a similar decrease: set #1 decreased of 104Ω and set #2 decreased of 103Ω. The average variance of the resistivity (not shown) of both sets was 65.2 Ω² and 80.3 Ω². As shown in FIGS. 6A, 6B, 6D, and 6E, all stripline sets showed similar resistive and reactive loads. The impedance of a capacitor is 1/(jωC), so the reactance or the imaginary part of the impedance changes as −1/(ωC) and both sets followed this law. The average reactance variance (not shown) of sets #1 and #2 were 49.5 and 36. FIG. 6C illustrates the measured return loss which is the difference, in dB, between forward and reflected power measured with the Agilent 16093/A binding post fixture and it does not vary with the power level at which it is measured (all the measurements were performed at 0 dBm). The Smith Chart results, illustrated in FIG. 6F (and is similar for both sets), suggest that all traces have a capacitance of approximately 10 pF estimated by the capacitance of a stripline. Because all the measurements shared the same Melinex® film, it can be deduced that all inks have very similar electrical permittivity of the dielectric, which is most likely the binder used for both the conductive and the dielectric inks.

### Example III

A set of simulations have been completed using HFSS (ANSYS Inc., Canonsburg), FEM electromagnetic simulation software to optimize the performance of the RTS leads, obtained from Ansys of Canonsburg, Pa.

The goal of this optimization is to simultaneously minimize the direct current (DC) resistance of the RTS lead during DBS treatment, while also minimizing the RF current at the tip of the lead which is exposed to the phantom during imaging. The lead should exhibit low resistance at DC to reduce ohmic power loss in treatment while exhibiting high impedance to RF current to reduce RF heating effects during imaging.

The simulation was performed at 64 MHz, the Larmor frequency at 1.5 Tesla. A parameterized model of a quadrature birdcage RF coil, ASTM phantom, and RTS lead were created as is illustrated in FIG. 7B. The coil in simulation is driven to create a rotating B₁ field. Variables are assigned to the length and conductivity of each segment of the RTS lead for optimization. However, it will be appreciated that other variables may be used for cost analysis and/or other number of RTS layers may be considered. A Quasi-Newton optimization is set with a cost function:

$$Cost = λ \cdot ( \frac{{l}_{1}}{{σ}_{1}} + \frac{{l}_{2}}{{σ}_{2}} + \frac{{l}_{3}}{{σ}_{3}} ) + \vert {J}_{lead} \vert$$

An initial simulation is run to determine the baseline value of the surface current J_{lead} on the tip of the RTS lead when in the presence of the rotating B₁ field. Starting values of σ=10 S/m and 1=0.2 m are used for all segments in the initial simulation. The lambda coefficient is used to evenly weight the optimization of both goals. HFSS uses an iterative solver and the FEM solution is obtained and followed by a second solution pass with a higher mesh density. This process repeats with increasing mesh density until a set convergence criteria is met. Convergence is typically set to compare the relative change in the S Parameters of the RF coil. In this case, the value of the RF current |J_{lead}| is used as the convergence criteria to ensure an accurate solution.

The result of the simulation, illustrated in FIG. 7A, shows that both the DC resistance and RF current can be reduced by a 3 layer RTS lead design. DC resistance was reduced by a factor of 3× and the RF current reduced by a factor of 100×. Therefore, the RTS leads will not have a lower resistivity than the present platinum-iridium wires (less than 1Ω), but will be designed to have a lower resistivity or less than the half contact impedance of the DBS electrodes (less than 500Ω).

### Example IV

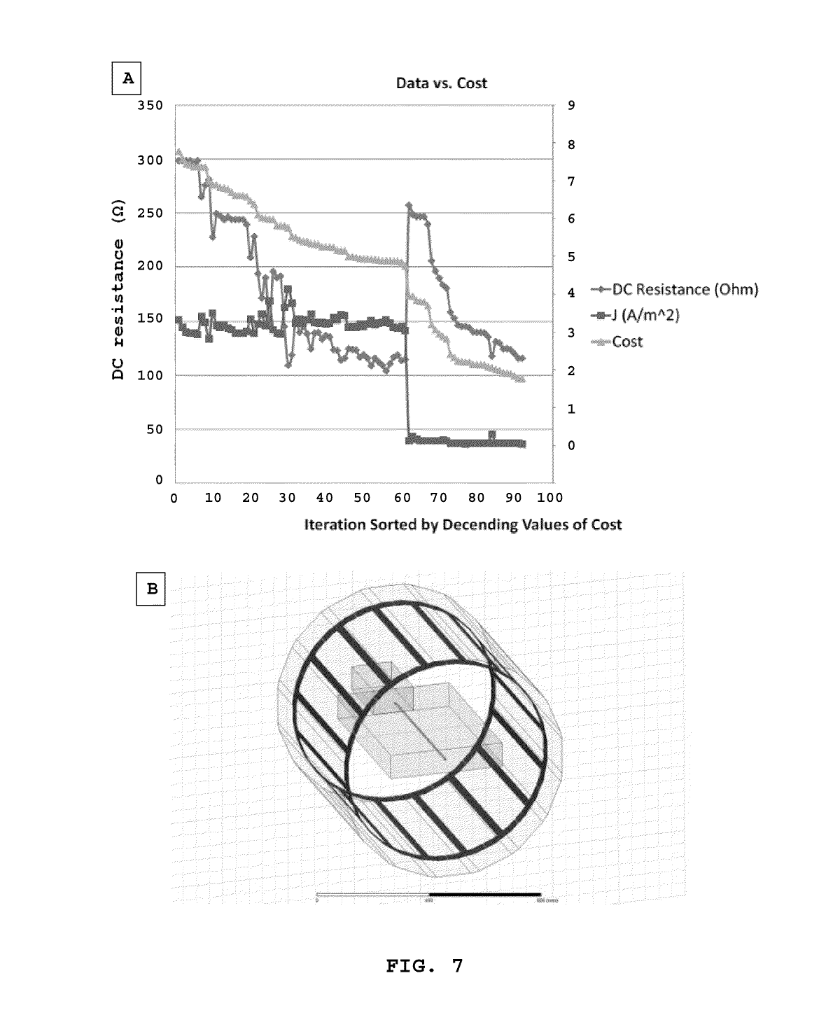

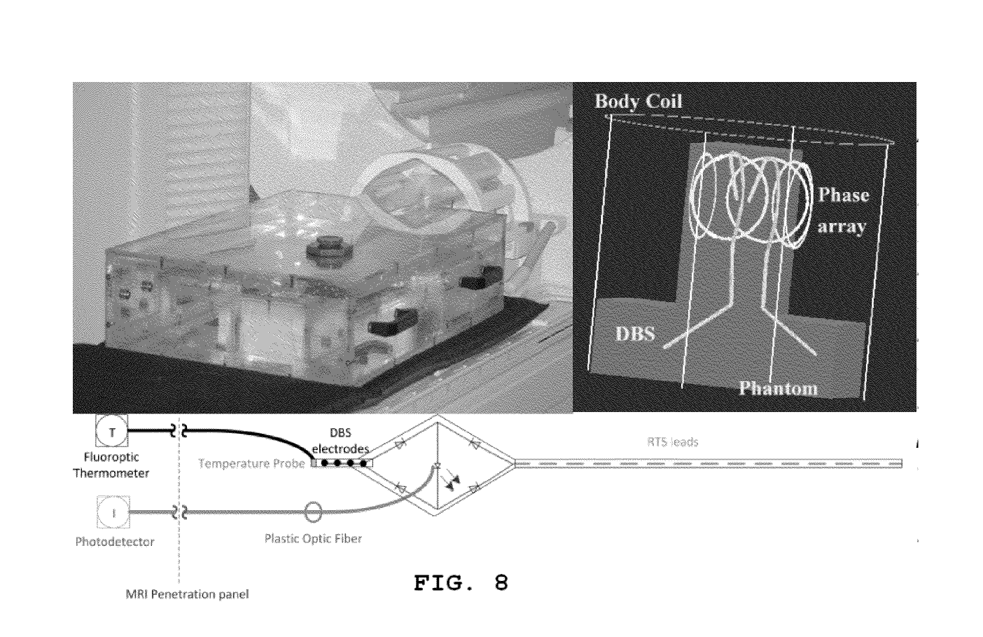

The RTS lead performance may be influenced by the presence of conductors near the head, such as receive phase array coils. Accordingly, the final RTS design is tested using electromagnetic simulations with a phase array receiver coil and a numerical model of the ASTM phantom with DBS implants as is illustrated in FIGS. 7B and 8. A transmit detunable birdcage body coil is modeled with 16 perfect electric conductors (PEC) rods closed by two rings. Reactive components are added to the geometric model in order to obtain a resonant coil at the Larmor frequencies of 64 MHz (1.5 T MRI) and 128 MHz (3 T MRI), but also at low field, including 21.3 MHz (0.5 T MRI) and 42.6 MHz (1 T MRI). The structure of interest is excited at a particular port with the load present in the same way as the physical coil.

Furthermore, two types of receive phase array coils are modeled: 12-channel array (one of the most common coil used in MRI scanners around the world) and 32-channel array coils, used for MRI-measurements. These two coils are geometrically positioned on a cylindrical surface centered on the ASTM phantom with RTS leads, which are illustrated in the upper-right figure of FIG. 8.

The use of the ASTM phantom allows that modeling of the entire RTS lead length up to the position of the implantable pulse generator (IPG) on the chest.

All lead materials and geometric dimensions are based on the Medtronic Activa electrode/lead set 3387 and extension 7496 discussed in greater detail below.

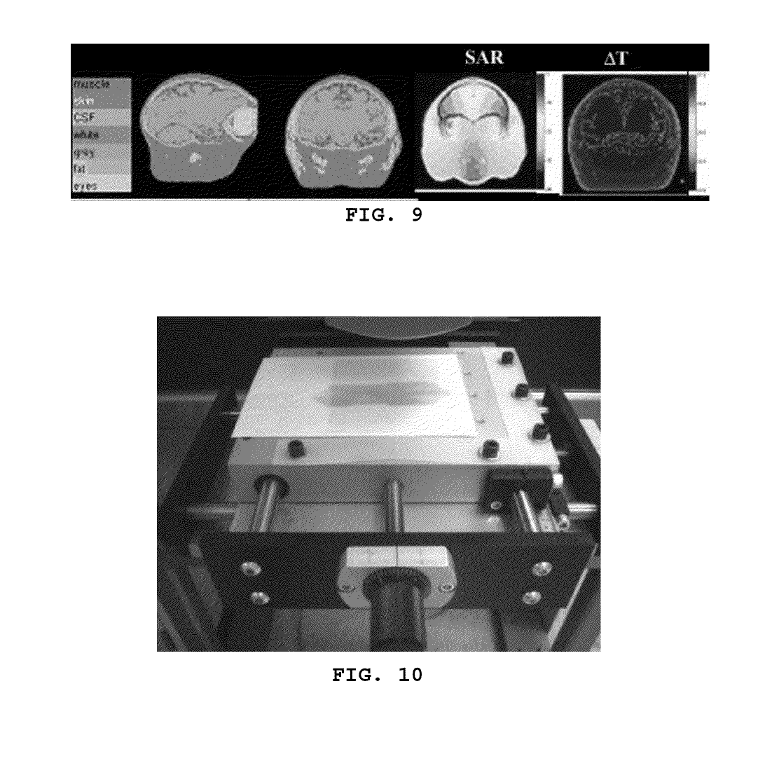

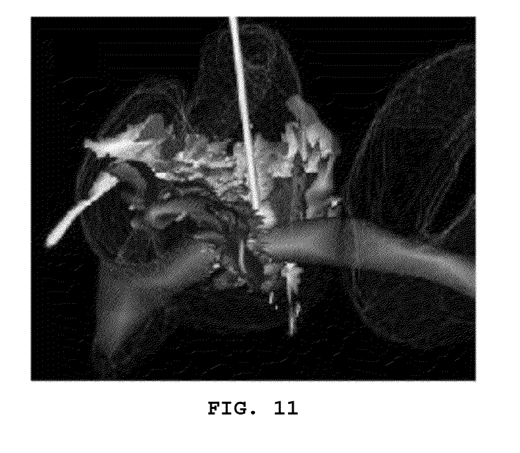

Likewise, in order to verify RTS simulations in monkeys, electromagnetic simulations are performed by: (a) importing the monkey segmented head model as illustrated in the two leftmost images of FIG. 9 into the EM-solver, (b) modeling a realistically shaped radiofrequency (RF) coil, (c) co-registering the intracranial electrode/lead into an anatomically precise monkey head model, and (d) performing data analysis and post-processing with Matlab. The electromagnetic fields and SAR are computed at 1×1×1 mm³ resolution using the FDTD and FEM electromagnetic solvers and the RTS lead solution may be recalculated.

A pacemaker leads testing setup follows that illustrated at the bottom of FIG. 8, including measurements of temperature using a fluoroptic optical thermometer and current using a photodetector. The calibration is done comparing the optical signal with the temperature measurements as has done in been done elsewhere (See, Nordbeck et al. “Measuring RF-induced currents inside implants: Impact of device configuration on MRI safety of cardiac pacemaker leads.” Magnetic Resonance in Medicine. 2009; 61(3):570-8.), therefore calibrating only for the currents responsible for Joule heating.

The simulations model up to 20 different lead paths, as taken from CTs scans of patient data.

Human model simulations can provide a systematic analysis of the effect of RTS leads on RF absorption in the human subjects head. In order to validate the leads design for use in non-human primates, a non-human primate head model (see the preliminary data set FIG. 9) is generated by means of semiautomatic segmentation of MRI data from one of the monkeys with DBS implants. The model is implemented with a 200 μm isotropic resolution and seven labeled anatomical structures including skin, eyes, fat, muscle, grey matter, white matter, and cerebrospinal fluid. Electrical and thermal properties are selected based on literature values. See http://www.fcc.gov/fcc-bin/dielec.sh and Bernardi et al. “Specific absorption rate and temperature elevation in a subject exposed in the far-field of radio-frequency sources operating in the 10-900-MHz range.” IEEE transactions on bio-medical engineering. 2003; 50(3):295-304.

### Example V

LCP-based RTS leads are manufactured closely following the specifications of the four electrodes Medtronic Activa DBS 3387 leads (lead kit for deep brain stimulation, Manual M927780A001, Medtronic Inc., Minneapolis, Minn.). The LCP-based RTS lead design is based on the PTF prototype built with conductive inks, where Carbon/Gold ink is formed as a suspension of CNPs/AuNPs encapsulated in a liquid resin called PTF filler, except that instead C/Au nanoparticles are mixed in the LCP filler. The same nanoparticles may be used as in the PTF prototype, since they provide optimal bonding binder: AuNPs of average size of 4-5 μm and largest spheres of approximately 14 μm and CNPs with average size less than 1 μm (such as are used in the CMI inks for the PTF prototype). LCP molecules have been shown to bond well with gold and LCP electrically conductive fibers are already commercially available (for example, Vectra A230D-3 from Ticona of Florence, Ky.).

To produce the LCP-based RTS lead, a company that specialize in plastics extrusion or the main manufacturer of LCP fibers is contacted to provide filaments with desired nanoparticle mixture in the molten thermotropic LCP state. The Au/LCP mixture is tested in an MRI to test for the presence of ferromagnetic metals that would produce imaging artifacts if the ferromagnetic metals are present. In order to avoid potential complications with clogging the capillary holes during extrusion, only large (approximately 100 μm) LCP fiber diameter are considered for manufacturing. Each fiber with the desired conductivity is aligned with a microscope and heat bonded to produce the final desired RTS fibers and are bundled and insulated with polyurethane as in the Medtronic DBS 3387. Polyurethane has excellent properties of moisture barrier, insulation and biocompatibility.

Manufacturing heat bounding used in plastic fibers or optical fiber mold laser welding used in optical fibers might alternatively be used to for final production.

Finally, the four bundles are mixed with non-conductive LCP for added strength and will be wrapped around an implant grade polyurethane sheathing (as in the Medtronic Activa leads DBS 3387) with the final outer diameter of 1.27 mm. Based on experience with PTF, it is not expected that the RTS fibers will produce any MRI artifacts.

### Example VI

Apart from the special polymer-based RTS leads, the rest of the implant is assembled in a clean room using commercially available parts similar to those of the Activa DBS system. In particular, the electrodes are made of platinum-iridium rings of 1.27 mm in diameter heat bonded to the RTS-LCP fibers on the distal end and connected to a MP35N connector like the DBS 3387 leads on the proximal end. The distal end has 4 electrodes that each are cylindrical in shape, have a length of 1.5 mm, have a spacing 1.5 mm, have a distance 10.5 mm, and have a distal tip distance 1.5 mm (matching the DBS 3387 leads). The proximal end has lead contact with a length of 2.3 mm, a spacing of 4.3 mm, a distance of 16.6 mm, and a Stylet handle length of 40.1 mm (again, matching the DBS 3387 leads). Similar design for the RTS extension is based on the 7496 (Extension Kit for Deep Brain Stimulation, Spinal Cord Stimulation, or Peripheral Nerve Stimulation, Manual UC199400538d EN, Medtronic) where the RTS leads described above are 51 cm long and connected to two quadripolar inline connectors: (distal) conductor wire diameter of 0.1 and lead entrance diameter of 1.47 mm and (proximal) contact diameters of 1.6/2.7 mm).

Finally, a commercially available MRI compatible stimulator is used, in the Model 7426 Soletra Neurostimulator by Medtronic. The entire new DBS system is sterilized before implantation by autoclaving or by gas (Ethylene Oxide, ETO), both which are sterilization procedures that have been approved for the Medtronic System. Validation testing (below) follows sterilization and device performance is documented.

### Example VII

The leads new DBS system is then tested following the performance specification of commercial leads in the premarket approval (PMA) of the Medtronic Activa System. Accordingly, the following tests are performed: (i) bench (ii) biocompatibility, and (iii) animal.

In both in vitro and in vivo studies, a set of non-clinical tests have been identified to support product development. The set of tests are applied to the complete and assembled leads made of: electrodes, RTS wires, connector and the complete system including the MRI compatible IPG (Medtronic Activa, using a commercial IPG). The bench tests are performed on all 20 prepared leads after sterilization. Destructive testing is performed last after all other testing has been completed and by recovering the leads from the euthanized animals.

The following battery of tests are performed:

The DC resistance between each electrode and the respective connector pin is verified as specified by the electromagnetic simulation and is target to be within 50% of the simulation values (in the DBS 3387, DC resistance must be less than 100Ω). All pins are tested to be either isolated or with a DC resistance >1 MΩ even after soaking in an isotonic saline solution for 10 days. In the primates studies, the MRI guidelines are followed for Medtronic deep brain stimulation systems (Medtronic Technical Note M929535A001) which specify an electrode contact resistance (that is, not considering the resistance of the RTS wires) of less 2 kΩ(open circuit) and greater than 250 Ω.

The leakage current during maximum voltage application (8.5V for the 7426 Soletra IPG) with maximum pulse width (210 μs) is tested to determine if it is less than 1 μA (ISO 14708-3:2008) after soaking 10 days in isotonic solution and before drying to simulate the effect of any body fluids on the lead body.

The tensile strength of each bond, RTS joint, etc. in the lead is determined, including the electrode to lead bond, lead to connector as well as composite lead tensile strength. The leads are subjected to tensile and flexural testing, which simulate the stress they may experience during the implant procedure, as well as after implant. Testing is performed on the dry lead and after the lead has been soaked in isotonic saline solution for 10 days to simulate any effects of body fluids on the lead body. The measured tensile strength of 10 fibers is compared before and after soaking and test for statistically significant change in the fiber's strength. The tensile strengths of each the leads, including the electrode to lead bond, lead to connector as well as composite lead on the proximal side of the connector are equal or more than 5N (ISO 5841-3).

It is expected that if they fail, the DBS leads would fail in proximity of the ring around the burr hole where they are subjected to higher stresses and the DBS extension tends to fracture at the neck or scalp area. Most lead or wire breaks occur between the extension cable and the DBS brain lead, which are located in the mastoid process. Resistance to mechanic fatigue of the conductors is tested. Therefore, faults in the electrical continuity of the DBS leads or the extension may occur with different loading conditions. Standard test methods (Mil-Std-883 Method 1010 Temp Cycle) will be followed designed to accelerate fatigue.

Testing for integrity of all joints, bonds, and so forth is performed to verify that the lead is leak-proof when immersed in an isotonic saline solution at 370° C. under physiological pressure of 150 mm Hg for 10 days. This is performed by electrical impedance spectroscopy (EIS), by checking that the impedance between any channel and the solution remains high (>100 MΩ) when the ends are not immersed in the solution.

In order to demonstrate that the lead can withstand the environment of the human body, the corrosion resistance of all conductors and electrode materials is tested. The leads are connected to the Medtronic IPG (7426 Soletra) and set to the worst case output parameters (185 Hz and 450 μs pulses) in an isotonic saline solution at 370° C. (but not the IPG) under pressure of 150 mm Hg for 10 days to test for environmental robustness. Digital imaging inspection with a microscope and performance testing measuring the EIS of the leads are performed and documented. Moreover, the overall resistance of the RTS fibers change is confirmed to be less than 50% from before and after this environmental exposure.

The performance of the planned stylet is evaluated during lead placement. The insertion and withdrawal forces are measured and that the stylet can be removed from the lead during the implant procedure without undue force exerted on the lead is tested and confirmed.

The performance of the anchoring sleeve packaged with the lead is evaluated. Testing assures that the lead is held securely in place and will not damage the lead body when the anchoring sleeve is sutured according to the implant manual by Medtronic.

Additionally, temperature changes along the RTS leads and on the electrodes are preferably within 2° C. from the baseline and might also be confirmed using the ASTM phantom (ISO 14708-3).

Finally, it is verified that the lead can be appropriately positioned with the epidural needle without damaging the lead.

### Example VIII

Over the past thirty years, several electro-physiological studies have been performed to examine the mechanisms underlying the effects of DBS using non-human primate models. These studies revealed neural responses elicited by DBS in intact neural circuits and provided direct means for examining how DBS modulates the basal ganglia thalamocortical circuits. DBS can modulate firing rate, normalize irregular burst firing patterns and reduce low frequency oscillations associated with the Parkinson's disease state.

The subthalamic nucleus (STN) was chosen as one of the DBS targets for three reasons. First, the STN receives a large quantity of GABAergic axonal terminal arborisations from the striatum that plays an important role in the planning and modulation of movement. Second, the STN receives a dopaminergic innervation from the nigra compacta that plays an important role in reward, addiction, and movement in monkeys. Third, stimulation of this structure affects the neuronal activity of the internal Globus Pallidus (GPi), the output nucleus of the basal ganglia involved in Parkinson Disease. The novel DBS system may stimulate any one of the standard target regions, specifically, the STN, the GPi or the ventral intermediate nucleus (Vim) of the thalamus. Other targets may include but not limited to the nucleus accumbens (NAc), dorsal striatum, lateral habenula, medial prefrontal cortex (mPFC), hypothalamus, and vmPFC-BG tract (ventromedial prefrontal-basal ganglia tract).

The standard and novel DBS systems are tested in the STN of healthy non-human primates. Experimentally, the efficacy of the novel DBS stimulation device will be accessed by reaction time (RT), movement time (MT), and movement accuracy (MA) during behavioral performance. Efficacy is estimated by comparing behavioral changes with the novel device “on” and “off” and the comparison between standard DBS and the novel. Stimulation is performed initially under the traditional high-frequency mode (that is, pulses of 130 Hz and 90 μs/phase pulse-width).

All the procedures below are reviewed and approved by the subcommittee on research animal care the Institutional Animal Care and Use Committee of MGH.

### Example IX

Biocompatibility experiments are performed on rats. Ten sprague-dawley rats around 6 weeks of age will be anesthetized with isoflurane/O₂/air (1-1.5% isoflurane) and placed in a stereotactic frame with a nose cone for continuous anesthesia. Briefly, the skull is exposed, the burr hole drilled, a RTS uncoated fiber in five rats (condition) or polyurethane implant grade cylinder of 100 μm matching diameter in five rats (control) is lowered slowly into internal STN (from Bregma: AP −2.6 mm, ML: 3.5 mm, DV: −7.5 mm). Two small bone screws are placed around the burr hole area as anchors for dental cement which will be used to hold the short RTS/Polyurethane fibers in place. The animals are observed until they awake from the anesthesia and then are housed in cages for three months. Then the rats are anesthetized using isoflurane (1-1.50) and perfused transcardially with PBS followed by 4% paraformaldehyde.

The rat brains are extracted and shipped to Charles River Laboratory of Wilmington, Mass. (CRL) for neurohistopathological assessment. CRL studies the biocompatibility of the proposed devices in accordance with FDA/CDRH/ODE blue book memorandum #G95-1. Hence, CRL focuses on functional damages due to the novel DBS lead surgical insertion and long term implantation. CRL performs the following tests on the brain specimens of the rats: anti-GFAP IHC (for astrogliosis), anti-NeuN IHC (general neuronal stain) and anti-IBA-1 IHC (a microglial marker). Finally, HSP70 will be tested for as a molecular marker of neurotoxicity.

While inflammatory and/or immune response of rats brain tissue due to AuNPs/CNPs might occur in principle, it is unlikely since the entire novel RTS fibers are normally (but not in these tests) coated with fluoropolymers and an implant grade polyurethane sheathing, which is the most common coating for stents, defibrillators, pacemakers and other devices permanently implanted into the body. Furthermore, spherical, anionic, of large 0.3-3 μm and 10-100 μm sizes, similar to the AuNPs used in the PTF, have been found to be completely non-toxic.

As will be described in greater detail below, monkeys are also imaged with CT/MRI to help clinically evaluate the effect of the new DBS leads. After thus testing, the animal brain specimens of monkeys around the STN/implant will be harvested and studied with scanning electron microscope (SEM) imaging and plasma-mass spectrometry (ICP-MS) at MGH to measure and report the AuNPs/CNPs concentration in the tissue.

The biocompatibility test is considered passed only if a final report shows that the new DBS leads perform as well as the commercially-available metal leads. If biocompatibility tests are not passed, the alternative approach is to sputter and electroplate platinum iridium onto the fibers with controlled thickness to match RTS resistance, since PtIr is already part of the standard DBS leads.

### Example X

Stimulation testing is performed in non-human primates. Four awake, behaving adult male Rhesus monkeys (Macaca mulatta) are treated with the DBS system. Before implanting the intracortical electrodes, each monkey is scanned using a Siemens 3 T MR scanner to acquire data for stereotactical registration. Each monkey is initially implanted with a plastic headpost and recording/stimulation channel, using ceramic screws covered by dental acrylic. This surgery is performed under general anesthesia by delivering isoflurane (1.5%)/N₂O (50%)/O₂ (50%). Temperature and CO₂ are monitored during anesthesia for the well-being of the animal and continuous saline solution will be administrated intravenously. Intravenous antibiotic and analgesics are administered intraoperatively. Postoperatively, the animals receive intramuscular injections of antibiotics and analgesics for three days and allowed to recover for two weeks.

The first step in the new DBS system implantation is to non-invasively localize the monkey's STN regions based on the animal's anatomical MRI scan. Localization of the STN in humans are routinely performed in humans during DBS surgery using similar electrophysiological and imaging methods.

The second step is the functional localization of the STN by microelectrode recordings at the target nucleus in the awake animals as they sit in a specially designed animal chair. The desired location for the target in the STN is centered in the dorsal sensory-motor region of the nucleus. The chair is designed to allow for the movement of the animals arms and legs while restraining the animals from reaching into the region of the recording chamber and hardware. Thorough mapping of the sensory-motor region of the STN may be achieved over the course of 1-2 weeks. The “sensory-motor territory” is localized using standard electrophysiological techniques. Data is sampled at 30 kHz, band-pass filtered between 300 Hz and 6 kHz and digitally recorded using microelectrodes (using Frederick Haer Inc., and Plexon data acquisition system). Neurons in this region change their firing rate/pattern with passive or active movements the contra-lateral limbs.

Next, two standard DBS systems (Medtronic Activa) in two monkeys and two novel DBS systems are inserted at the same targeted sites bilaterally and fixed to the interior of the recording chamber using bone cement. Prior to surgery, the animals are trained at begin each trial to fixate a small point on a touch-screen monitor. Then, a “Bull's Eye Target” appears at one of four positions in the corners of the monitor. The animal receives the highest reward if it accurately touches the small center of the target and progressively smaller rewards with less accurate movements. The reaction time (RT) and movement time (MT) and movement accuracy (MA) behavioral performance are recorded. The monkeys are monitored by a camera mounted in the room and an eye-tracker. The behavioral performance at different onset times is studied to confirm that the new leads are delivering the stimulus, by comparing RT, MT and MA on the two monkeys with RTS in comparison to the two monkeys with the standard DBS set.

The resulting slides are analyzed with digital imaging techniques and the analysis will focus primarily on cell death or shrunken cell bodies present around the implant or in the substantia nigra. The histology of the tissue surrounding the polyurethane versus RTS fibers and the representative digital images will be compared, specifically discarding the effect of potential confounds, such as mechanical trauma of insertion; long-term inflammation, neuronal response; and implant-induced injury. Furthermore, the voxels around the implant are segmented to extract scar borders and thinning as done to compare the lesion volumes, cross sectional areas and thickness in the two conditions. The histological statistical image processing is performed using a Bayesian analysis on the co-registered (commercial versus RTS) segmented images and the Cohen's kappa coefficient is estimated to measure the agreement of the raw digital images from CRL. Finally, every extracted new DBS implant is analyzed with SEM for biostability of the insulator by using this state-of-the art SEM analytical technique to look for signs of cracks and stress in each of the LCP conductive fibers.

### Example XI

Herein, a novel MR Conditional lead based on resistive tapered stripline (RTS) design is further detailed, a high scattering technology that allows for decreased tissue heating while maintaining low lead resistivity for continuous current injection. The RTS design attenuates the antenna performance and reduces the induced current at the electrode by introducing scattering within the implant. The optimal RTS design parameters have been studied by electromagnetic simulations, showing a 37-fold reduction in induced current at the electrode when compared to a metallic wire. Experimental temperature measurements with a 3 T MRI system and battery testing with a commercial DBS implantable pulse generator were performed. Measurement results showed that the heating near the electrode of the RTS prototype was less than 2° C. compared to 9° C. obtained with a commercially available DBS lead. There were no significant differences in battery consumption between the two leads over a one-month period. The potential impact of RTS design is highly significant to medical research and patient health care as the new technology may allow an increasing number of patients with active implants access to standard diagnostic medicine.

As noted above in the background section, implanted medical devices such as cardioverter-defibrillators, pacemakers, spinal cord stimulators, and deep brain stimulators have become well-adopted therapeutic options to treat a large range of medical ailments and contribute to improve quality of life. Many patients with implanted devices may benefit from MRI, which is the diagnostic tool of choice for monitoring structural changes in the body as well as diagnosing many common disorders including cancer, cardiovascular disease and trauma. Additionally, functional MRI is becoming more dominant in assessing brain function and cognitive disorder. However, approximately 300,000 patients with implanted or partially implanted medical devices are denied MRI each year because of safety concerns. A major concern when performing MRI examinations in patients with electrically conductive implants is the high induced currents (“antenna effect”) along the conductive lead exposed to the radiofrequency (RF) waves of the MRI. These high currents flow into the tissue at the point of contact with the lead (i.e., the distal electrodes) causing a large amount of RF energy to be absorbed in the tissue which, in turn, causes surges in temperatures that may lead to serious injury. Temperature increases of up to 25° C. near DBS electrodes have been measured with an in-vitro gel phantom at 1.5 Tesla MRI. Additionally, increases of up to 30° C. were measured in a pig at 9.4 Tesla. Still yet, two cases of serious, permanent neurological injury after MRI exposure at 1.0 Tesla in patients with DBS implants have been reported. In the most severe case, when manufacturer guidelines were not followed, a patient with bilateral DBS implants underwent MRI and suffered an edema near one of the implants with a consequent paralysis. The accidents associated with implants and MRI are expensive to society due to treatment that often includes hospitalization.

Certain implantable devices—defined as “MR Conditional”—have been shown to pose no known hazards in the MR environment when operated with specified conditions. Nevertheless, there are drawbacks even for MR Conditional devices. For example, the conditions under which a patient with an implanted MR Conditional DBS system can safely undergo MRI are extremely restrictive and exclude the most commonly used transmit body coils, the 3 T systems, and the state-of-the-art MRI multichannel transmit coils, which represents a significant constraint to the use of MRI for many patients with DBS.

To solve the issue of RF-induced heating without interfering with device performance, several proposals have been made to modify the design of the implant such as introducing RF chokes, modifying the materials of the lead, or utilizing special geometrical paths for the wire. Herein, a new type of lead based on RTS technology is utilized. The RTS design can be best understood by recalling oceanic science, where an area of study is the prevention of standing waves (“clapotis”). Special constructions reinforced with wide rubble-mound beams break up wave energy over some distance, preventing the formation of clapotis. Similarly, tapered dielectric structures can break up or scatter RF energy due to their unique frequency response characteristics.

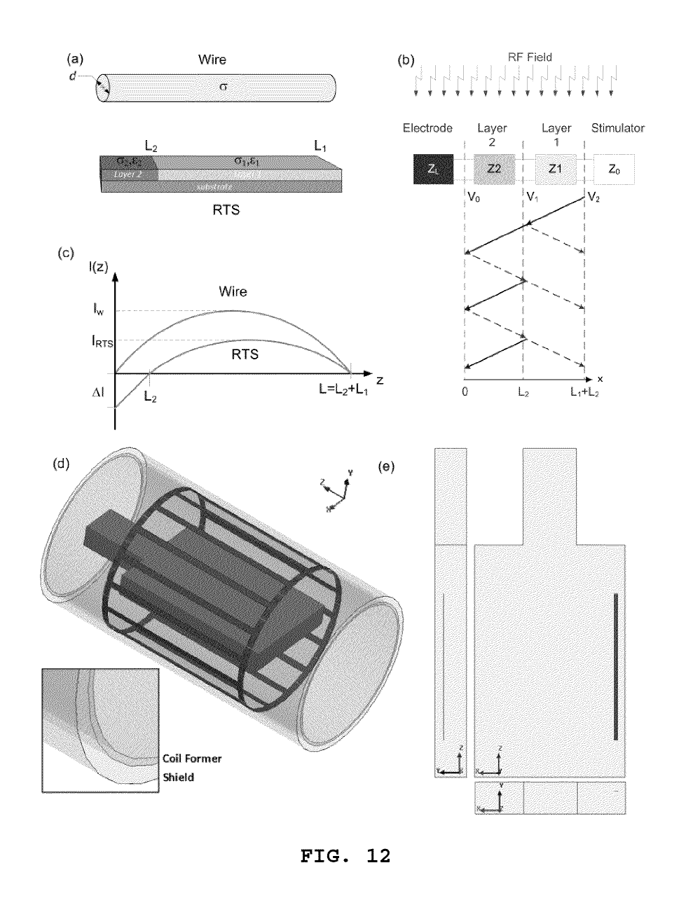

In FIG. 12A, a two-section stripline-based design is presented with an abrupt variation of electrical conductivity along its length. Contrary to a standard electrically homogeneous cylindrical wire, this design can break up the induced current along the lead caused by the MRI RF coil. Subsequently, RF induced current along the RTS lead would be more heterogeneously distributed as illustrated by FIG. 12B and significantly reduced at the distal electrode. This in turn causes a reduction of energy absorption in the tissue surrounding the electrode.

There are various advantages to this RTS design. Numerical simulations and experimental testing confirmed that the RTS design allows for “RF-transparency” while maintaining proper conductivity as not to affect battery performance. Additionally, as shown from previous studies, a Polymer Thick Film (PTF) design may produce lower T1, T2 and T2* MRI artifacts since in general it contains less metal than conventional implants. Finally, this RTS design does not require any external physical device such as a RF choke. RF chokes are difficult to attach to an implant wire because the dimensions of a choke are larger than the typical dimension of the wire. In addition, chokes disrupt the mechanical characteristics of an implant wire, which should remain flexible. Although there are extremely miniaturized RF chokes, these devices are more prone to burn because of the physical dimensions, thereby causing additional surgeries to explant and re-implant a device just to replace a burnt RF choke.

### Example XII

The evaluation of safety from RF-induced heating in patients with implanted medical devices undergoing MRI is based on several testing strategies and tools, including pre-clinical (experimental, computational, and animal testing) as well as clinical testing. Experimental testing includes measuring temperature changes near the device while implanted in gel-type material that simulates electrical and thermal characteristics of the human body. Additionally, computational modeling has been increasingly used to complement experimental testing, as it allows for extensive, cost-effective and systematic analysis of several variables that can influence the amount of current flow into an implant and the amount of energy absorbed by surrounding tissue.

A computational model was used to evaluate several possible electrical and geometrical configurations of the RTS lead to minimize the absorption of energy and the temperature increase at the distal electrode. For the results illustrated in FIG. 12C, the model included a clinical MRI RF transmit coil loaded with a model of a gel-filled phantom and the implanted lead as illustrated in FIG. 12D. Simulations were utilized to determine the values of electrical conductivity and length for a two-section RTS design that was then used to build a prototype for experimental validation. The prototype was then implanted in a gel-filled phantom and tested in a 3 T MRI system. Both simulations and measurements confirmed that the RTS design allowed the lead to be “transparent” to the incident RF-field, that is to say, the presence of the lead did not significantly affect the RF fields present in the empty phantom.

### Example XIII

In other contexts, RTS design has been successfully introduced in landmine detection to improve the antenna performance. By contrast, in the context of medical imaging, the RTS design aims to decrease the antenna performance and resulting induced currents along the wire.

Simulations were performed that included a realistic MRI birdcage transmit coil tuned at 128 MHz, an ASTM phantom, and a model of a realistic PTF conductive inks that allowed for a physically realizable solution.

The parameter used to evaluate the power-absorbed inside the phantom near the distal electrode was the specific absorption rate (SAR) averaged over 10 g of tissue (i.e., 10 g-averaged SAR or 10 g-avg.SAR). The SAR is the dosimetric parameter used in RF-safety guidelines; it is measured in W/kg and it is directly proportional to the initial increase of temperature inside a volume exposed to RF energy. Current guidelines of the International Electrotechnical Commission (IEC) limit the SAR over the whole-body in Normal Operating Mode to 2 W/kg and the maximum 10 g-averaged SAR to 10 W/kg.

A model was created with the FEM electromagnetic solver ANSYS HFSS v15.0 and circuit solver ANSYS Designer v8.0. The dimensions and material properties of the coil, lead, and phantom are listed in Tables I and II, below:

TABLE I

| Geometry | Dimension | |
|---|---|---|
| Coil Diameter | 610 | mm |
| Coil Length | 620 | mm |
| Coil Shield Diameter | 660 | mm |
| Coil Shield Length | 1220 | mm |
| Coil and Shield Thickness | 0.1 | mm |
| Coil Ring/Rung Width | 25 | mm |
| Coil Former Inner Diameter | 590 | mm |
| Coil Former Wall Thickness | 10 | mm |
| Lead Length | 40 | cm |
| Lead Width | 0.5 | mm |
| Lead Thickness | 15.7 | μm |
| Contact Length | 1.5 | mm |
| Contact Width | 0.5 | mm |
| Lead Substrate Width | 10 | mm |
| Lead Substrate Thickness | 25 | μm |
| Lead Insulation Width | 5 | mm |
| Lead Insulation Thickness | 25 | μm |

TABLE II

| Material | Value |
|---|---|
| Copper (Coil and Shield) Conductivity | 5.8 10⁷ S/m |
| PMMA (Coil Former) Permittivity | 3.0 |
| Platinum (Contact) Conductivity | 9.3 10⁶ S/m |
| ASTM Phantom Conductivity | 0.47 S/m |
| ASTM Phantom Permittivity | 80 |
| Kapton HN (Lead Substrate) Permittivity | 3.5 |
| DI-7502 (Lead Insulation) Permittivity | 2.5 |
| Conductive Ink (Lead) Permittivity | 5.0 |

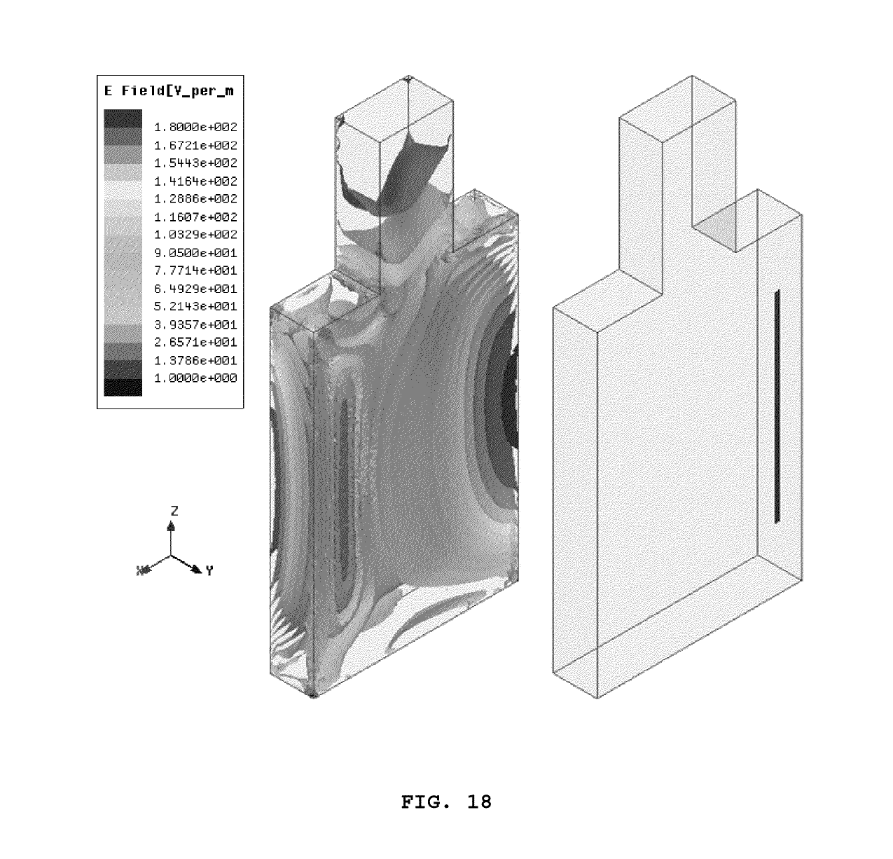

The S-parameters at frequencies above and below the Larmor frequency were generated using the inductive interpolation method described in Tronnier, V. M., Staubert, A., Hahnel, S. & Sarem-Aslani, A. Magnetic resonance imaging with implanted neurostimulators: an in vitro and in vivo study. Neurosurgery 44, 118-125 (1999). The initial value of the tuning capacitance was estimated and adjusted until the unloaded coil system was tuned to 128 MHz. The correct coil mode was identified by first tuning the coil to a resonant mode and then plotting the resultant current distribution in HFSS to ensure a sinusoidal distribution of current in the rungs of the coil as per Li, H., Li, L. & Toh, K.-A. Advanced topics in biometrics. (World Scientific, 2012). The value of capacitance for each identical capacitor located around the rings of the high-pass birdcage was found to be 13.5 pF. Each capacitor also includes an approximated 0.05Ω of equivalent series resistance which was estimated. The input voltage to the coil was adjusted in the circuit simulator to set a whole-body averaged SAR within the phantom of 2.0 W/kg (i.e., Normal Operating Mode for MRI systems). The input voltage was found to be approximately 115V. The complex magnitude of the electric field was calculated in the phantom under these conditions for the purpose of determining the area of peak electric field. FIG. 18 shows a 3D plot of the magnitude of the electric field at the Larmor frequency f₀=128 MHz at a power level yielding whole-body SAR of 2 W/kg within the phantom. This field plot helps to visualize the target locations of peak values of electric field, where the lead was placed. An asymmetry in the field due to the circular polarization and the shape of the ASTM phantom can be seen with peaks located laterally across the anterior and posterior sides of the phantom. Once this area of peak electric field was located, a model of a conductive ink stripline lead was placed in this location as shown in FIG. 18. For each lead design simulation, the 10 g-avg. SAR was computed at a point 0.1 mm from the anterior face of the lead contact in the direction of the positive Z-axis.

The simulation workflow and EM model utilized for experimental validation are identical to those previously described for the optimization study except for the dimensions of RF transmit body coil which was modeled after the Siemens Skyra system, the 3 T MRI system used for the temperature measurements.

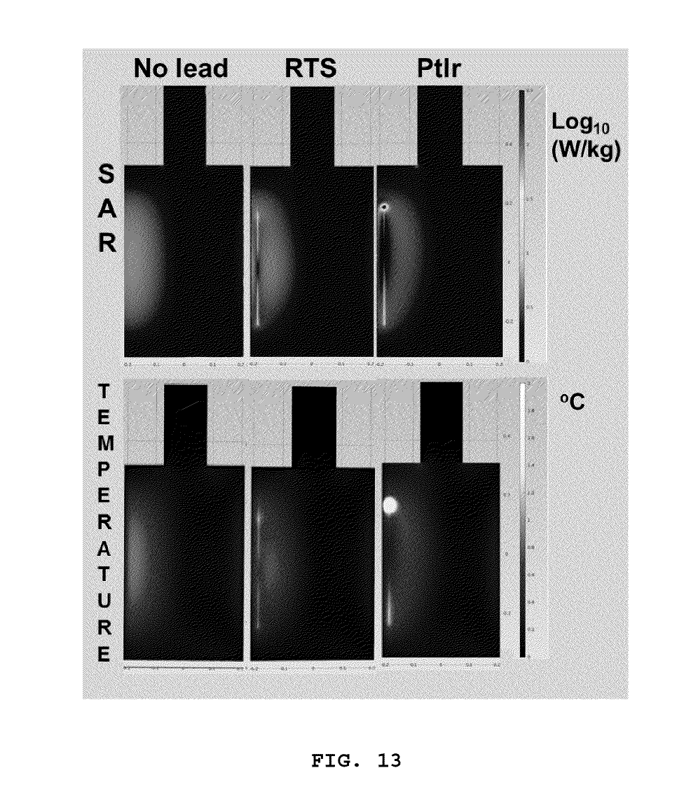

FIG. 13 shows the 10 g-avg.SAR and temperature maps in the model of the phantom without implant, with the RTS profile that was selected for prototype manufacturing, and with a single-electrode conductive PtIr cylindrical wire, respectively. The SAR and temperature maps, which are plotted throughout the plane containing the lead show similar results between the RTS lead and the case without the implant, with temperature changes below 1° C. during a 15 minute exposure at a whole-body SAR of 2 W/kg (Normal Operating Mode). By contrast, the single-electrode metallic cylindrical wire generated a temperature change of 64° C. over the same exposure time.

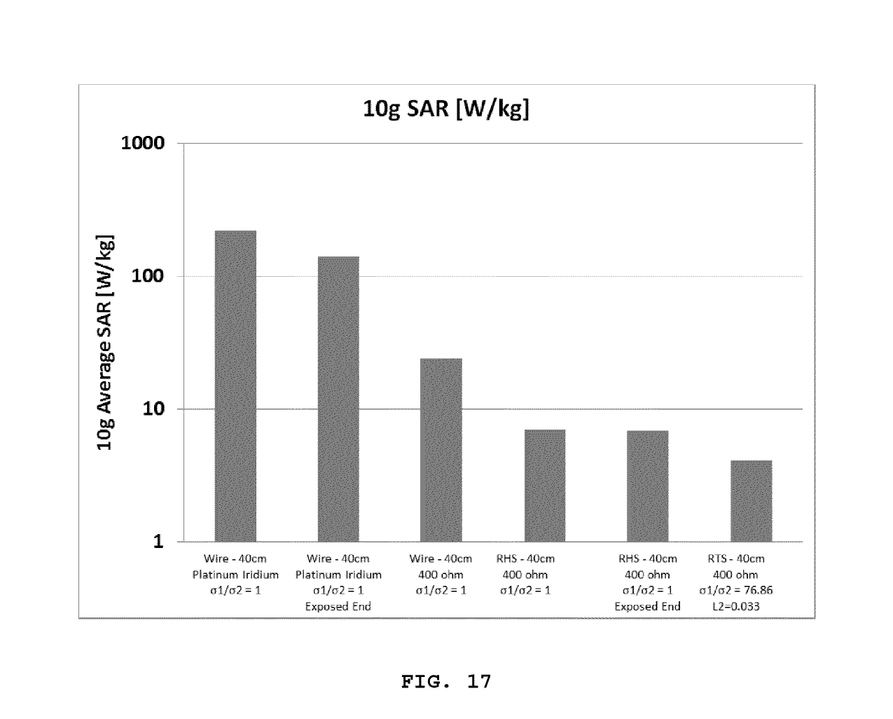

The numerical simulations showed that standard DBS lead implants act as an antenna during the RF transmit period of the MRI scan, picking up the induced electric field and inducing a high RF energy in the volume surrounding the exposed electrode tip. Simulations were performed using Finite Element Method, which allowed for a high spatial resolution at the distal electrode, where the highest electric field was observed. The accuracy of the simulations was subsequently validated against in-vitro temperature measurements in a gel-filled phantom, discussed below. Notably, the simulations and manufacturing were performed with the distal electrode exposed only on a single side, although additional simulations included the case of two exposed tips providing the data illustrated in FIG. 17. The single side opening is the most realistic case, because the proximal end is typically connected to an impulse generator, which usually includes RF chokes necessary to protect the internal circuitry from damage by external large RF fields. Compared to standard leads in both simulations and in-vitro testing, the RTS showed that the proposed design successfully reduces the inductance of the lead and breaks the current along the lead, reducing the amount of energy absorbed at the distal electrode and the related temperature changes inside the gel-phantom.

From a review of FIG. 17, it can also be seen from the simulations, that ink thickness plays a role in the RF induced currents. As the printed traces are thinner and less conductive, their ability of conducting RF currents decreases even further by the skin depth law. A homogenously conductive thin-flat design decreased the current density at thicknesses less than the skin depth as illustrated in FIG. 17.

### Example XIV

To initially model the RTS design, the RTS design contained two discrete sections of variable conductivity and length, connected in series. Three limitations were used to minimize the optimal design search including: a) total length fixed to 40 cm, b) conductivity of the proximal section higher than the distal section; and c) total low-frequency resistance of the lead equal to 400Ω.

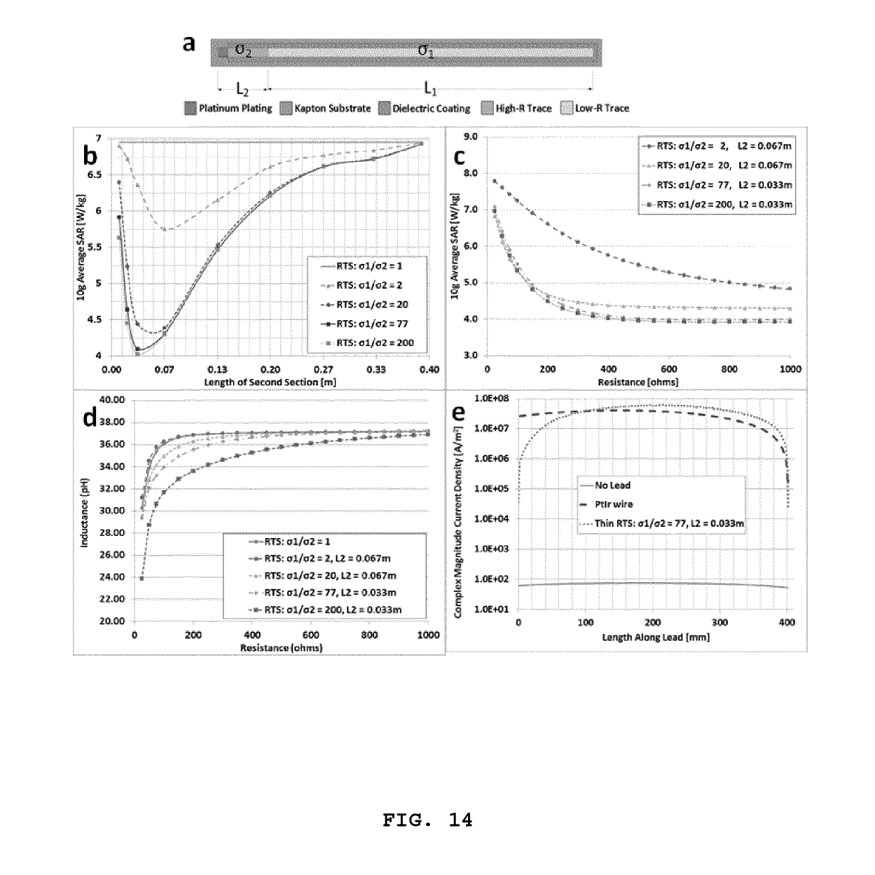

With reference being made to FIG. 14A, the values of the RTS design's conductivity (that is, σ₁ and σ₂) and length (i.e., L₁ and L₂) that were ultimately used to build a prototype were derived by a series of numerical simulations. The total length for all the designs was L=40 cm, to match standard lead designs per the search limitations identified above. As illustrated in FIG. 14B, the specific length of each of the two sections of the RTS lead was shown to non-linearly affect the 10 g-averaged SAR at the distal electrode. This analysis was performed by sweeping each trace across the same values of length, but with different values of the ratio in conductivity (σ₁/σ₂) between the two sections of the lead. FIG. 14C shows the 10 g-averaged SAR in the phantom near the distal electrode with a resistance sweep from 0Ω to 1 kΩ for several RTS designs. The RTS lead reduced the 10 g-averaged SAR across the entire range of possible resistances. The simulations showed that an increase in conductivity ratio between the two sections corresponded to a decrease in 10 g-average SAR at the distal electrode. For example, the optimal RTS (that is, σ₁/σ₂=200) plateaued at 400Ω with a value of 4.02 W/Kg, whereas the design with σ₁/σ₂=2 showed a 10 g-avg.SAR of 5.75 W/Kg at 400Ω. With reference being made to FIG. 14D, the SAR reduction was due to a lower inductance of the RTS design that corresponded to a shorter equivalent antenna length and lower induced currents. As confirmed by the simulations, the RTS design was characterized by a reduced current at the distal electrode of over two orders of magnitude. Based on the constraints on the electrical conductivity of the ink used for manufacturing, one RTS configuration was selected to build a prototype with the following characteristics: σ₁=1.968·10⁶S/m, σ₂=25.61·10³S/m (i.e., σ₁/σ₂=76.86), L₁=0.367 m, and L₂=0.033 m. The total resistance for the RTS design was chosen to be R=400Ω, five times less than the maximum electrode/tissue impedance of 2 kΩ allowed by even older IPG models. As shown in FIG. 14B, the 10 g-SAR of this configuration was expected to be very similar to the best performance of the RTS lead with ratio σ₁/σ₂=200 (i.e., 4.1 W/kg vs. 4.02 W/kg, respectively).

### Example XV

A RTS lead prototype was constructed, based on optimal parameters derived from simulation, in order to experimentally test the proposed concept. The RTS lead was manufactured using two different conductive PTF inks: silver and carbon-based. The carbon-based ink, which is significantly lower in conductivity, was mixed with the silver based ink to adjust the conductivity of the second section to the desired value indicated by the simulations. The length of the most conductive layer was given by the conductivity of the silver ink and a length that allowed for a less conductive layer with target ratio σ₁/σ₂=77, giving the overall resistivity of 400Ω. Notably, the resistivity of both the carbon traces and silver electrodes is flat between 100 Hz to 200 MHz. The 400Ω resistance was well within the range of current commercial IPGs considering that the contact electrode/tissue resistance is usually below 1 kΩ. The tolerance for the resistivity was 5% and the tolerance for length was 50 μm.

The primary feature of the RTS is the very sharp change of conductivity between the two sections. While this discontinuity can be easily modeled computationally, possible issues may arise in a real prototype, because the two structures would need to be built using different inks and it is possible that the two inks may not perfectly overlap. Once the prototype was built, experimental testing confirmed that it was in fact possible to generate such a discontinuity. The physical overlap of the two structures was about 50 μm long.

The RTS lead was manufactured using two different commercially available conductive ink materials. The first ink, 479 SS (Electrodag, Acheson LTD., Kitano, Japan) is a silver (Ag) based PTF ink and was fixed to a specified resistivity of 0.02 Ω/sq./mil according to its datasheet. This ink was used in the higher conductivity section L₁. The second ink 423 is a carbon (C) based PTF ink, which has significantly lower in conductivity compared to Ag based PTF inks of 42 Ω/sq./mil according to its datasheet. The final layer L₂ was fabricated by chemically mixing the two PTF inks to adjust the conductivity of the second section to the value prescribed by the simulations, which was constrained to be within the conductivity values of the Ag and C-based PTF inks.

The prototype used for the experimental measurements was built using silver to reduce the initial costs. Silver is not a biocompatible material and future versions of the lead will need to use more expensive biocompatible materials (such as, for example, gold).

Further, uncertainty studies showed that the permittivity of the binder used in conductive inks is of interest for local SAR estimation. Binders serve to bind together the nano-particles of the material, ensure the necessary viscosity for proper transfer of the ink from the press to the substrate, provide adhesion to the substrate, and contribute to the drying speed and resistance properties of the ink. The relative permittivity of binders varies from 2 to 15 or higher in composites that significantly exhibited the RTS effect; no RTS effect was found with binders with relative permittivity of vacuum for the RTS.

### Example XVI

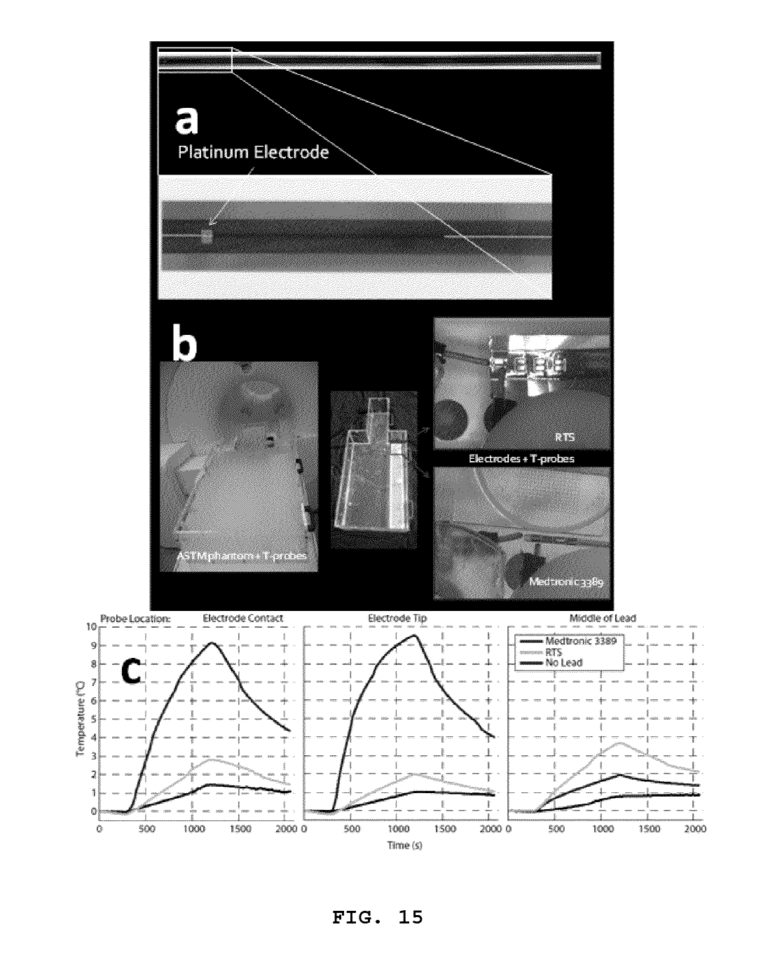

The RTS prototype was tested using a 3 T MRI system as depicted in FIG. 15. In the same session, the RTS prototype and a commercially available DBS lead (Medtronic 3389) were placed in a scaffold inside a gel phantom. The phantom shell was made of Plexiglas and filled to a volume of 24.6 L with a PAA gel (product number 436364, Sigma Aldrich Co. St. Louis, Mo.) mixed in an aqueous solution (distilled water, conductivity less than 1 mS/m) and with NaCl-reagent grade, >99% pure (S9888, Sigma Aldrich Co., St. Louis, Mo.). The ratio of the mixture was 1.32 g NaCl and 10 g PAA for each 1 L of water. The mixture created a semisolid gel that approximated the dielectric constant and thermal convection of human tissue. A plastic scaffold with adjustable posts (i.e., plastic screws, bolts and washers) was placed on the far right side of the phantom and was utilized to consistently position system components (i.e., electrode lead and temperature probes) within the phantom.

Three MRI-compatible fiber optic temperature probes were used to record the temperature profiles of set points along the lead. A 3 T MR system (Skyra, Siemens, Erlangen, Germany) was programmed to deliver high RF energy exposures corresponding to First Level Control mode in a 15-minute MRI scan. The RTS prototype was tested against a commercially available lead (3389, Medtronic Inc.), for comparison with previous studies.

Temperature increase due to exposure to the MRI RF field was measured using fluoroptic probes as depicted in FIG. 15B. The temperature increase near the distal electrode of the commercial lead was about 9° C. higher than the baseline level of the phantom without lead, in line with previously published results. The temperature increase of the RTS lead was 3° C. around the electrode contact, and less than 4° C. for the probe located in the middle of the lead as depicted in FIG. 15C. This was in line with the energy distribution predicted by the simulations that suggested a decrease of current at the distal electrode and a higher current along the lead due to the scattering characteristics of RTS technology. For reference, the baseline temperature increase of the phantom without implant was 1.5° C. at the location corresponding to the distal electrode and 1° C. at the location near the middle of the lead as again illustrated in FIG. 15C. In order to enhance the signal to noise ratio of the measurements, the testing was performed with high levels of RF power, namely a whole-body SAR reported by the machine of 4 W/kg. Most sequences used in MRI systems are characterized by a whole-body SAR of less than 2 W/kg (i.e., “Normal Operating Mode”). Given the linear relationship between SAR and temperature, the corresponding maximum temperature increases in Normal Operating Mode (i.e., whole-body SAR of 2 W/kg) would be less than 4.5° C. with the 3389 lead and less than 2° C. with the RTS. For reference, the level of temperature increase suggested by the ISO standard for patients with implantable neurostimulators is 2° C., to which the experiments demonstrate the RTS lead abides.

### Example XVII

Additionally, because the RTS prototype was built with a resistance of 400Ω, which is higher than that of a standard PtIr lead, there were concerns over the performance of the lead with respect to battery consumption.

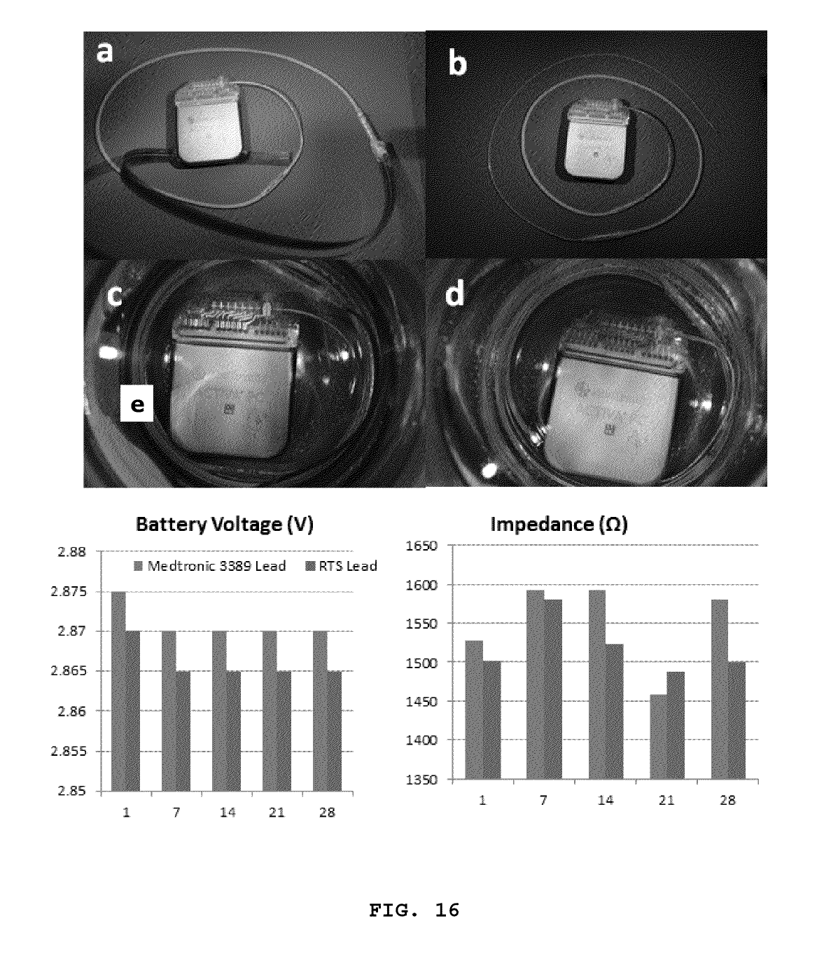

For this purpose, a preliminary comparative test was performed by connecting the RTS prototype and a commercial lead (3389, Medtronic Inc, Minn.) to a commercially available Implantable Pulse Generator (IPG) (Activa PC, Medtronic Inc., MN) as illustrated in FIGS. 16A and 16B. The IPG, extension and lead were placed in a quart of deionized water mixed with saline solution to simulate in-body tissue impedance as illustrated in FIGS. 16C and 16D. The IPG was turned on for a total of four weeks and voltage and inductance were measured approximately once every week as logged in FIG. 16E.

The testing was performed using the following parameter settings for Medtronic Activa PC IPG: i) single lead (contact 0) set to negative (−), ii) case set to positive (+ and contacts 1, 2 and 3 were all turned off), iii) unipolar cathodic pulse train set to an amplitude of 2V, a frequency of 130 Hz and a pulse width of 90 μs. For the Medtronic 3389 lead the following procedure was used: the extension wire was connected and fastened (with set screw) to the Activa PC IPG, the 3389 lead was connected and fastened to extension wire; the connection site was covered with silicon wrap and sealed on both ends with non-dissolvable suture, which is the same procedure utilized in the operating room to produce a water tight implant. For the RTS lead, the following procedure was used: the extension wire connected and fastened (with set screw) to Activa PC IPG and the RTS lead connected to (contact 0) on the extension wire with silver epoxy (8331, MG Chemicals, Surrey. B.C., Canada) and insulated with super glue; the connection site was covered with silicon wrap and sealed on both ends with non-dissolvable suture. A Digital multimeter was used to check for conductivity between RTS contact and extension wire.

For both leads, the IPG, extension and lead were then placed in a quart of deionized water and saline solution was added to deionized water until impedance measured 1500Ω. A hand held Medtronic Physician programmer was utilized to activate the IPG to test for therapeutic impedance. The IPG was turned on and left on for a total of four weeks; during this time the IPG was tested once a week for two measures, 1) an oscilloscope was used to check and measure emitted pulse train in saline solution, and 2) the hand held programmer was used to monitor changes in therapeutic impedance. Saline was added to the water if impedance dropped below 1500 Ω.

The results demonstrate that the high resistivity of the RTS lead does not compromise the power consumption of the Medtronic Activa IPG. Furthermore, finding that the RTS lead battery level profile was the same as the Medtronic lead provides confidence that the low power dissipation material used in the RTS is suitable for chronic implantation since it will not reduce the advertised nine years battery life.

While several embodiments have been described and disclosed, it will be apparent to those skilled in the art that other changes can be made as well. Therefore, the present invention is not to be limited to just the described most preferred embodiments. Hence, to ascertain the full scope of the invention, the claims which follow should also be referenced.

We claim:

1. A lead for a deep brain stimulation system in which the lead is adapted for electrical communication with a neurostimulator and extends to a distal tip for attachment to at least one electrode, the lead comprising:
a lead wire comprising a liquid crystal polymer including conductive particles dispersed therein.

2. The lead of claim 1, wherein the liquid crystal polymer comprises the structure:

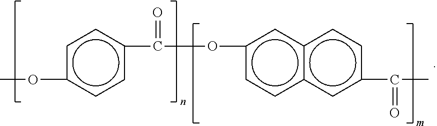

3. The lead of claim 1, wherein the liquid crystal polymer comprises polyesterpolyarylate fibers.

4. The lead of claim 1, wherein the conductive particles are nanoparticles.

5. The lead of claim 4, wherein the nanoparticles are gold nanoparticles.

6. The lead of claim 5, wherein the gold nanoparticles have an average diameter of 4 to 5 μm.

7. The lead of claim 4, wherein the nanoparticles are carbon nanoparticles.

8. The lead of claim 7, wherein the carbon nanoparticles have an average diameter of less than 1 μm.

9. The lead of claim 1, wherein the conductive particles are melt polymerized with the liquid crystal polymer to disperse the conductive particles throughout the liquid crystal polymer.

10. The lead of claim 1, wherein the lead wire with has abrupt variations in resistance over a length of the lead wire.

11. The lead of claim 1, wherein the lead is approximately 1.3 mm in diameter.

12. The lead of claim 1, wherein, when the lead is implanted in a patient and subjected to radio frequency waves in an MRI device, the lead does not heat more than 2 degrees Centigrade in an applied field of 3 Telsa.

13. The lead of claim 1, further comprising an insulating outer coating on the lead wire.

14. The lead of claim 13, wherein the insulating outer coating is polyurethane.

15. The lead of claim 13, wherein the lead comprises multiple bundles, in which each bundle includes a lead wire that with an insulating outer coating, and wherein the multiple bundles are packaged together in a single lead.

16. The lead of claim 15, wherein each of the multiple bundles are received in additional liquid crystal polymer which has an insulating sheathing.

17. The lead of claim 1, wherein the lead wire is adapted to conduct direct current for deep brain stimulation treatment, while remaining substantially transparent in clinically-applicable MR environments.

18. A deep brain stimulation device comprising a neurostimulator and an electrode, wherein the lead of claim 1 places the neurostimulator and the electrode in electrical communication with one another.

19. An MR-compatible lead, the lead comprising:
a lead wire comprising a liquid crystal polymer including conductive particles dispersed therein.

20. A method of making a lead, the method comprising:
mixing a liquid crystal polymer and conductive particles to form a mixture in which the conductive particles are dispersed in the liquid crystal polymer; and
forming a lead wire from the mixture.

21. The method of claim 20, wherein the step of forming the lead wire from the mixture involves extruding the mixture to similarly orient fibers of the liquid crystal polymer in a direction of extrusion.

22. An MR-compatible lead, the lead comprising:
a lead wire comprising a polymer including a conductive phase dispersed therein.
